(19) United States
(12) Patent Application Publication
(10) Pub. No.: US 2015/0322071 A1
(43) Pub. Date: Nov. 12, 2015

(54) PROCESS FOR PREPARING TRIAZOLE PYRIMIDINE COMPOUNDS AND INTERMEDIATES THEREOF

(71) Applicant: BRIGHTGENE BIO-MEDICAL TECHNOLOGY (SUZHOU) CO., LTD, Suzhou, Jiangsu (CN)

(72) Inventors: Jiandong YUAN, Suzhou (CN); Qiao JIANG, Suzhou (CN); Xiang LI, Suzhou (CN)

(73) Assignee: BRIGHTGENE BIO-MEDICAL TECHNOLOGY (SUZHOU) CO., LTD, Suzhou, Jiangsu (CN)

(21) Appl. No.: 14/647,738

(22) PCT Filed: Dec. 4, 2013
(86) PCT No.: PCT/CN2013/088517

§ 371 Date: May 27, 2015

(30) Foreign Application Priority Data
Dec. 6, 2012 (CN) 201210518595.0
Jul. 23, 2013 (CN) 201310310506.8

(51) Int. Cl. C07D 487/04; C07D 317/44; C07D 405/12; C07C 269/06

(52) U.S. Cl. CPC C07D 487/04; C07C 269/06; C07D 317/44; C07D 405/12; C07C 2101/08

(57) ABSTRACT

A method is disclosed for preparing Ticagrelor using the compounds as represented by intermediates of formula (2), formula (1-b), formula (1-c), formula (1-d) and formula (1-e), wherein the definition of R is as described in the description, and also disclosed is a method for preparing the intermediate of formula (2) and the use thereof in the preparation of Ticagrelor.

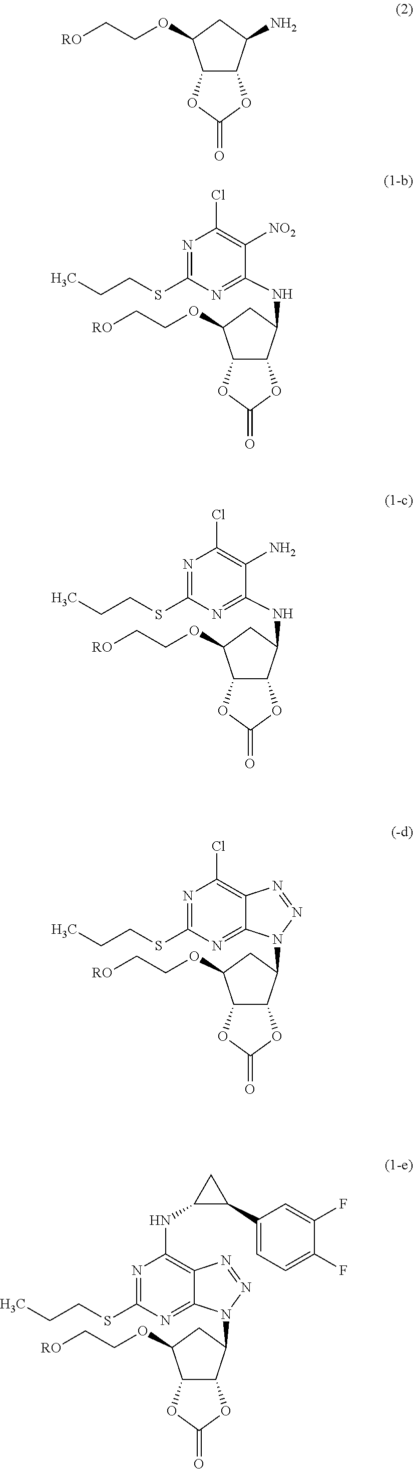

### FIELD OF THE INVENTION

The present invention relates to a process for preparing a new anticoagulant, Ticagrelor and new intermediates useful for manufacturing Ticagrelor.

### BACKGROUND OF THE INVENTION

Ticagrelor (Trade name: Brilinta, CAS: 274693-27-5), (1S,2S,3R,5S)-3-[7-{[(1R,2S)-2-(3,4-difluorophenyl)cyclopropyl]amino}-5(propylthio)-3H-[1,2,3]-triazolo[4,5-d]pyrimidin-3-yl]-5-(2-hydroxyethoxyl)cyclopentane-1,2-diol, has the following chemical structure:

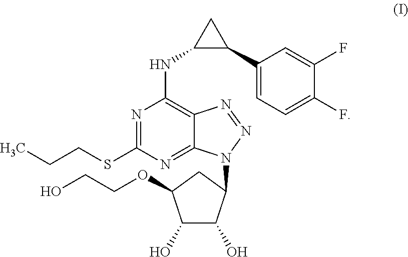 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

Ticagrelor is a new oral selectively small molecular anticoagulant developed by AstraZeneca. It reversibly interacts with the platelet P2Y12 ADP-receptor to prevent signal transduction, platelet activation and aggregation. Due to the rapidly onset of action after oral, Ticagrelor can significantly improve acute coronary syndrome in patients with cardiovascular. Compared with Clopidogrel, Ticagrelor has anti-platelet aggregation activity and does not require metabolic activation. In another way, Ticagrelor has the similar anti-platelet aggregation activity but with less side effect and has a more broad application prospects.

At present, processes of preparing Ticagrelor disclosed by foreign patents mainly are the following:

WO9905143 has disclosed a process for preparing Ticagrelor as shown in the following scheme 1:

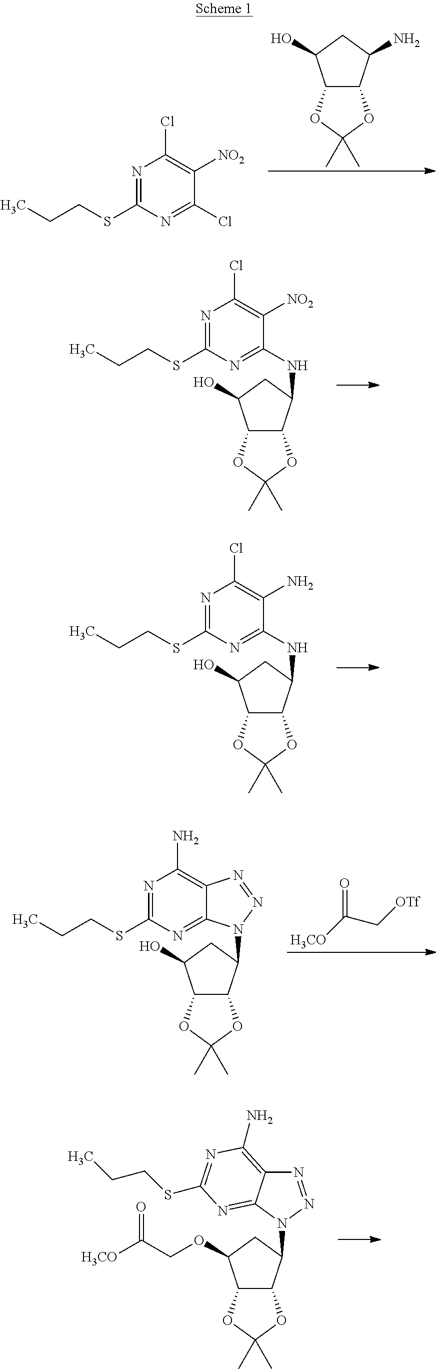

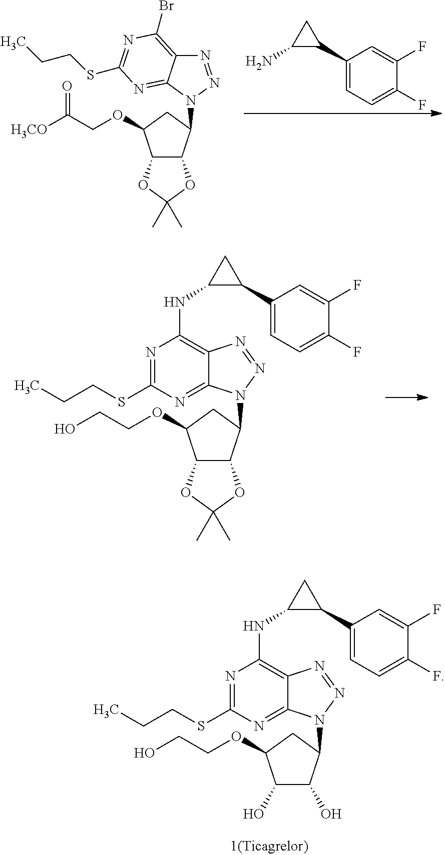

This synthetic path is very long and the reaction conditions are harsh, which greatly affects the product yield and is not suitable for industrial production.

WO0192263 has disclosed a process for preparing Ticagrelor as shown in the following scheme 2:

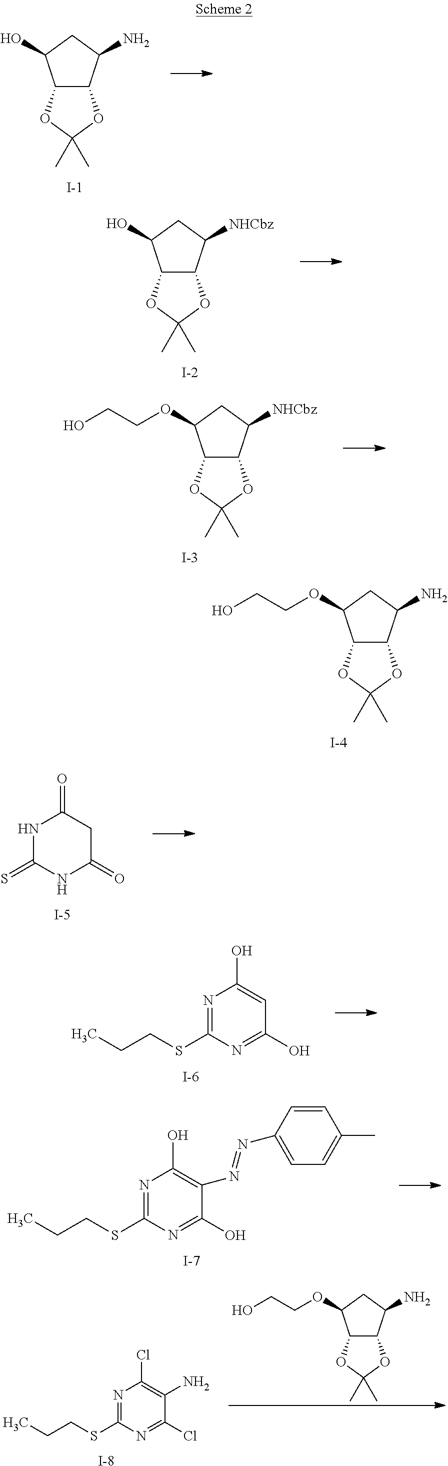

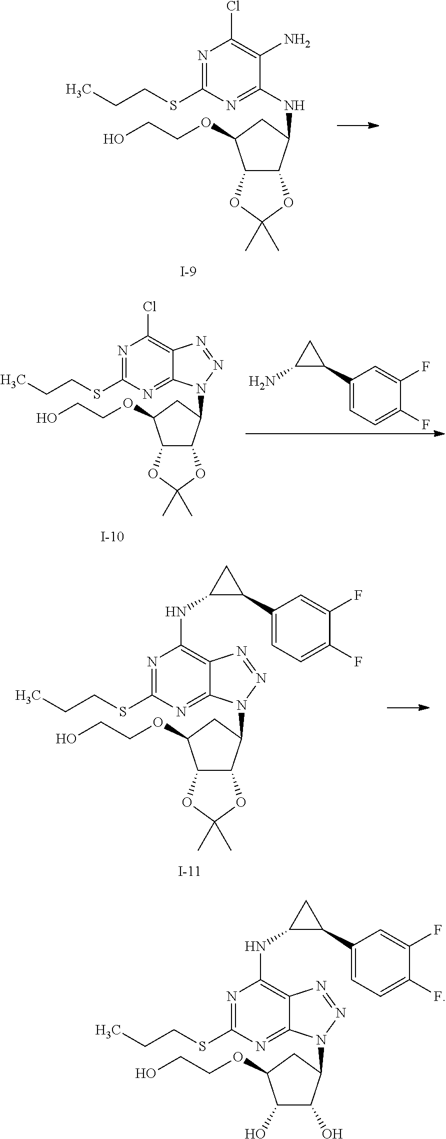

In this process, intermediates of formula I-8, I-9, I-10 and I-11 are isolated as oil, liquids, making these intermediates much more difficult to handle and purify. In some steps in the above process the purity and yield of the intermediates are all greatly affected. Finally, the product quality of Ticagrelor can not be guaranteed.

WO2011017108 has disclosed another process for preparing Ticagrelor as shown in the following scheme 3:

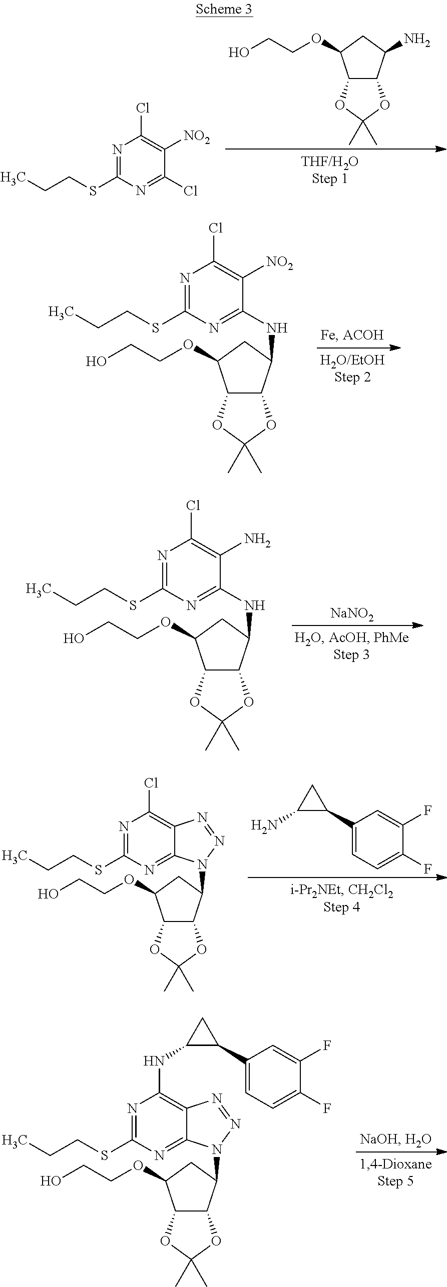

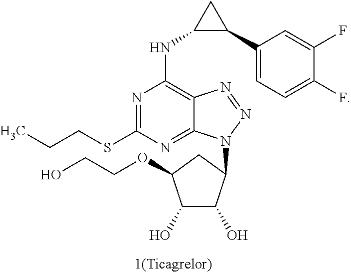 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

Comparing this process with that described in WO0192263, the amino of pyrimidine ring is substituted with nitryl, which making the first step much more easily to react. Also the reaction condition has been optimized and the synthesis path has shortened. However, this process still exists the following defects: the intermediates are isolated as oil, liquids and are difficult to handle and purify. All these defects affect the quality of Ticagrelor.

### DETAILED DESCRIPTION OF THE INVENTION

The present invention is directed to provide an improved process for the preparation of Ticagrelor. In this process, the reaction conditions are mild, the purities of the intermediates are easily to control, and the product quality and yield can be ensured.

In order to achieve the above object, the present invention adopts the following technical scheme:

A process for preparing Ticagrelor, comprising:
1) coupling the compound of formula (1-d) with a compound of formula (3) or salt thereof to provide a compound of formula (1-e):

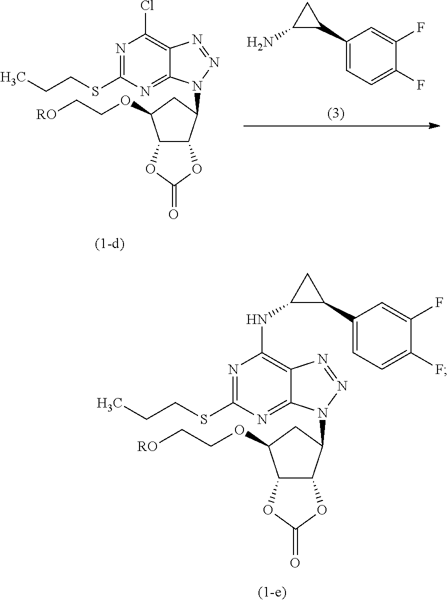

and,
2) deprotecting a compound of formula (1-e) to provide Ticagrelor:

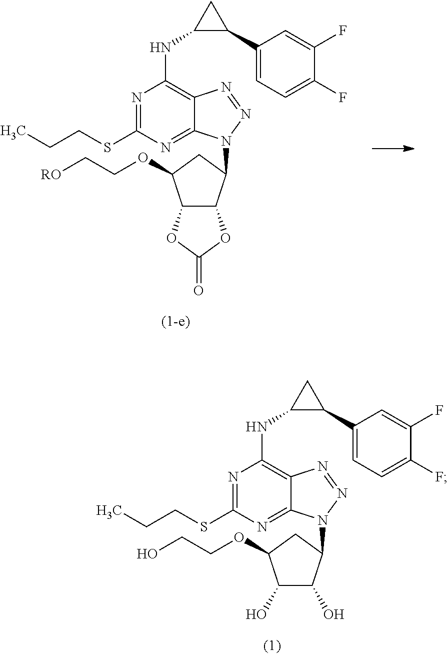

wherein, R is H or hydroxyl protecting group. Preferred, what said hydroxyl protecting group is selected from the group consisting of silicyl, triphenylmethyl or substituted triphenylmethyl, benzyl or substituted benzyl, alkoxylmethyl or methyl substituted by alkoxyl, acyl, allyl or substituted allyl or alkoxycarbonyl.

Further preferably, what said hydroxyl protecting group is selected from the group consisting of tert-butyldimethylsilyl, tert-butyldiphenylsilyl, triphenylmethyl, benzyl, p-methoxybenzyl, 2-tetrahydropyranyl, methoxymethyl, 2-ethoxyethyl, acetyl, benzoyl, pivaloyl, allyl, tert-butyloxycarboxyl, carboxybenzyl, 9-fluorenylmethyloxycarbonyl or alloc.

In the above step 1) a compound of formula (1-e) can be prepared by coupling the compound of formula (1-d) with a compound of formula (3) or salt thereof in the presence of a suitable base (such as tertiary amine, such as a tri(C1-6) amine, for example N,N-diisopropylethylamine) and a suitable solvent (such as dichloromethane) and at a suitable temperature (such as a temperature in the range 10-40° C., preferred 20-30° C.).

In the above step 2), when R is H, this process also comprises deprotecting the carbonate of the compound of formula (1-e) to obtain Ticagrelor (the compound of formula (I)) in the presence of a suitable solvent (for example polar solvent, such as dioxane, 1,3-dioxolane, tetrahydrofuran and so on).

When R is hydroxyl protecting group, this process also comprises deprotecting the carbonate of the compound of formula (1-e) as described above to obtain the compound of formula (1-f):

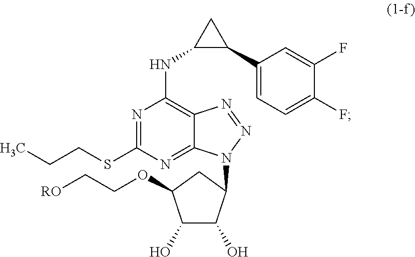

Further, comprising deprotecting the compound of formula (14) with the common method to obtain Ticagrelor in a suitable solvent and in the presence acid;

Or, when R is hydroxyl protecting group, R in the compound of formula (1-e) can be deprotected at the same time with the deprotecting of the carbonate of the compound of formula (1-e) in the presence of base.

Further, a compound of formula (1-d) can be prepared by cyclization of a compound of formula (1-c):

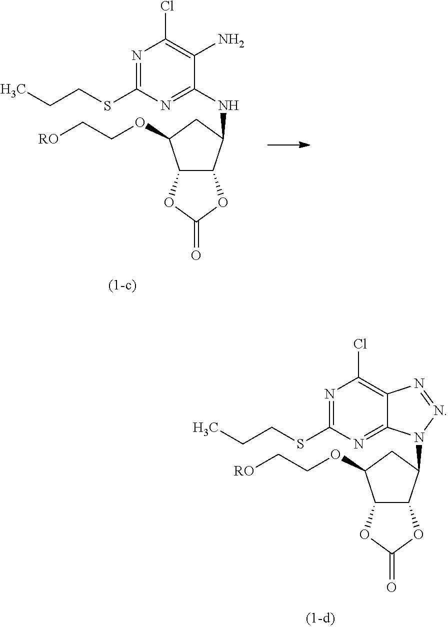

A compound of formula (1-d) can be prepared by reacting a compound of formula (1-c) with an alkali metal nitrite (such as NaNO₂) or an organic nitrite (for example isoamylnitrite) in the presence of a suitable acid (such as acetic acid) and a suitable solvent (such as toluene or a mixture of toluene and water) and at a suitable temperature (such as a temperature in the range −10° C. to 15° C., for example 20° C. to 30° C.).

In a further aspect, said the compound of formula (1-c) can be prepared by reducing of a compound of formula (1-b);

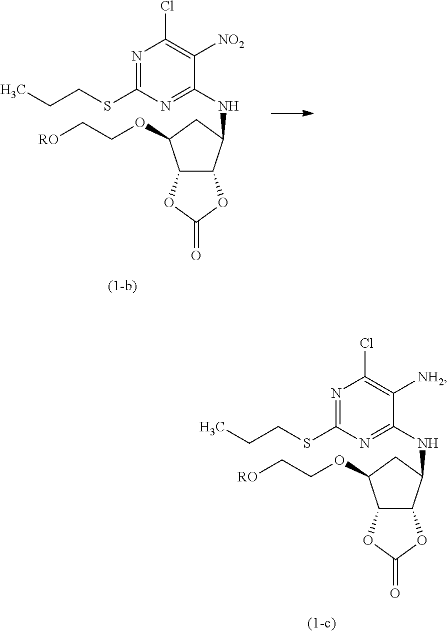

Wherein, the reducing reaction can be conducted in the presence of the suitable reductant, such as using active metal (for example Ferrum pulveratum) as the reductant and water as the reacting solvent; or using sulfide as the reductant and so on; Preferred, the suitable reductant is active metal and the reacting solvent is water; During the process of the reducing reaction, little acid and a mixture of water and organic solvent can be added to the reacting mixture.

Or, a compound of formula (1-c) can be prepared by reacting a compound of formula (1-1) with a compound of formula (2) or salts thereof:

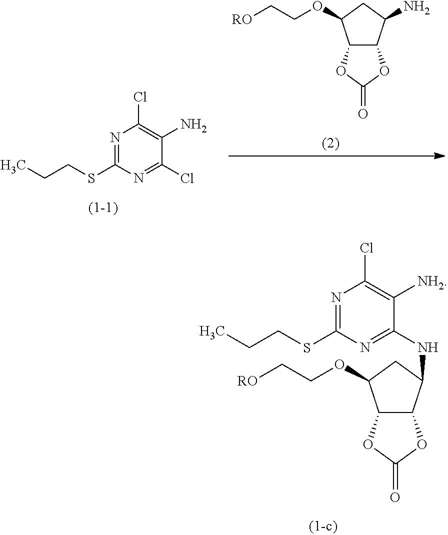

Said reacting a compound of formula (1-1) with a compound of formula (2) or salts thereof can be in the presence of suitable solvent (such as N,N-dimethylformamide, dimethyl sulfoxide or N-methyl pyrrolidone) and at suitable temperature (such as a temperature in the range 70° C. to 110° C., for example 85° C. to 95° C.).

Wherein, R in the compound of formula (2) is hydrogen or hydroxyl protecting group; what said hydroxyl protecting group is preferably selected from the group consisting of silicyl, triphenylmethyl or substituted triphenylmethyl, benzyl or substituted benzyl, alkoxylmethyl or methyl substituted by alkoxyl, acyl, allyl or substituted allyl or alkoxycarbonyl.

A suitable salt of the compound of formula (2) can be a salt of a mineral or organic acid. Suitable mineral acids include hydrochloric, hydrobromic, hydroiodic or sulfuric acid. A suitable organic acid is for example an organic achiral acid such as acetic acid, trifluoroacetic, oxalic, maleic acid or p-toluenesulfonic acid, or an organic chiral acid such as L-tartaric acid or dibenzoyl-L-tartaric acid

In a further aspect, said the compound of formula (1-b) can be prepared by reacting a compound of formula (1-a) with a compound of formula (2) or salts thereof:

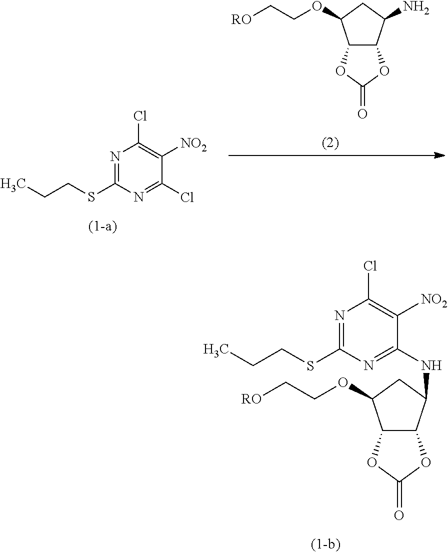

for example in the presence of a suitable solvent (for example ether, such as tetrahydrofuran) and at a temperature in the range −10° C.˜20° C. Preferably, said the reaction is conducted at a temperature in the range 0-10° C. Wherein said a salt of a compound of formula (2) is a salt of a mineral or organic acid. Suitable mineral acids include hydrochloric, hydrobromic, hydroiodic or sulfuric acid. Suitable organic acid is for example an organic achiral acid such as acetic acid, trifluoroacetic, oxalic, maleic acid or p-toluenesulfonic acid, or an organic chiral acid such as L-tartaric acid or dibenzoyl-L-tartaric acid.

The compound of formula (1-1) can be prepared by reducing of a compound of formula (1-a). CN1128801C has disclosed the preparation of the compound of formula (1-1) which is incorporated to the present invention by reference.

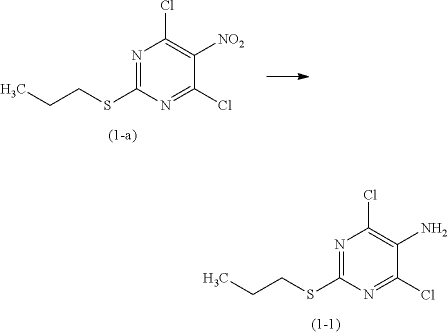

Another object of this invention is to provide a compound of formula (2) or salts thereof:

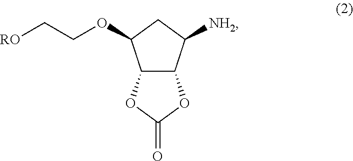

Wherein, R is H or hydroxyl protecting group. Wherein said hydroxyl protecting group is selected from the group consisting of silicyl, triphenylmethyl or substituted triphenylmethyl, benzyl or substituted benzyl, alkoxylmethyl or methyl substituted by alkoxyl, acyl, allyl or substituted allyl or alkoxycarbonyl

In a further aspect, wherein said hydroxyl protecting group is selected from the group consisting of tert-butyldimethylsilyl, tert-butyldiphenylsilyl, triphenylmethyl, benzyl, 2-tetrahydropyrane, methoxymethyl, 2-ethoxyethyl, acetyl, benzoyl, pivaloyl, allyl, tert-butyloxycarboxyl, carboxybenzyl, 9-fluorenylmethyloxycarbonyl or alloc.

Wherein said a salt of a compound of formula (2) is a salt of a mineral or organic acid. Suitable mineral acids include hydrochloric, hydrobromic, hydroiodic or sulfuric acid; Suitable organic acid is for example an organic achiral acid such as acetic acid, trifluoroacetic, oxalic, maleic acid or p-toluenesulfonic acid, or an organic chiral acid such as L-tartaric acid or dibenzoyl-L-tartaric acid.

Another object of this invention is to provide a method for preparation of a compound of formula (2) or salts thereof, comprising:

1) protecting a compound of formula (2-b) with cyclic carbonate to obtain the compound of formula (2-c):

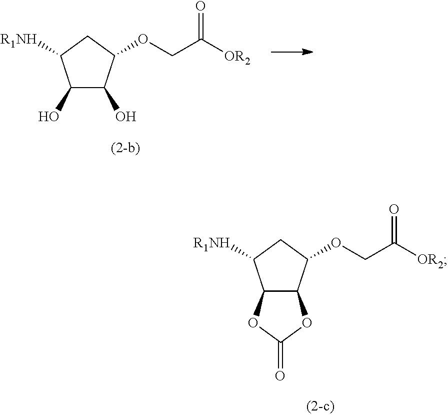

2) reducing the compound of formula (2-c) to obtain the compound of formula (2-d)

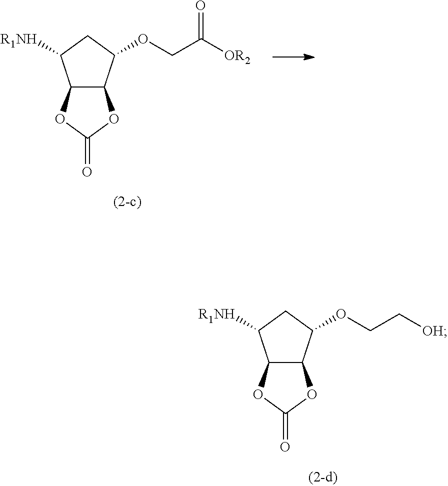

3) deprotecting the compound of formula (2-d), or further to protect the hydroxyl group to obtain the compound of formula (2):

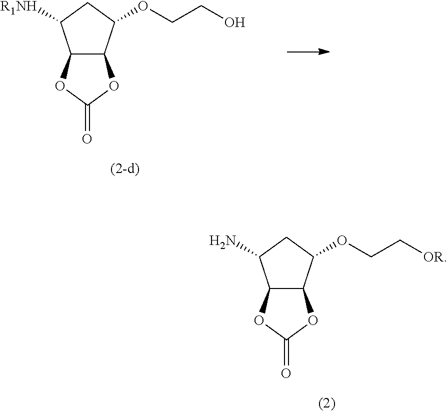

Wherein, R₁ is amino protecting group, preferred R₁ is carboxybenzyl (Cbz), tert-butyloxycarboxyl (BOC), benzyl (Bn), p-methoxyphenyl (PMP) or 9-fluorenylmethyloxycarbonyl (FMOC). Further preferably, R₁ is carboxybenzyl (Cbz), tert-butyloxycarboxyl (BOC), benzyl (Bn).
Wherein, R₂ is C₁₋₄ alkyl, preferably R₂ is methyl, ethyl, propyl or butyl. Wherein said R is H or hydroxyl protecting group, and what said hydroxyl protecting group is defined as above.

Further, the compound of formula (2-b) can be prepared by deprotecting of the compound of formula (2-a):

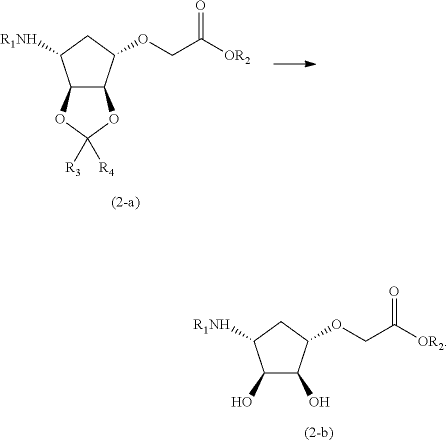

The compound of formula (2-a) is commercially available, or can be prepared as the method described in CN1938290B. Wherein, R₃ and R₄ are C₁₋₆ alkyl. Preferably R₃ and R₄ are both methyl. The preparation of the compound of formula (2-b) by deprotecting of the compound of formula (2-a) is in the presence of a suitable solvent (for example alcohols, such as ethanol) and acidic condition.

The step 1) above, protecting a compound of formula (2-b) with cyclic carbonate to obtain the compound of formula (2-c) is in the presence of a suitable base (for example organic base, such as pyridine, triethylamine) and a suitable solvent (for example non-polar organic solvent, such as dichloromethane, chloroform) and at a temperature in the range −100˜0° C. Preferably the compound of formula (2-c) is prepared by reacting a compound of formula (2-b) with triphosgene or N,N′-carbonyldiimidazole.

In step 2) above, the compound of formula (2-c) can be prepared by reducing the compound of formula (2-d) with a suitable borohydride (for example an alkali metal borohydride, such as lithium borohydride), lithium aluminiumhydride or DIBAL-H in a suitable polar solvent (such as tetrahydrofuran) and at a suitable temperature (such as at a temperature in the range −20˜10° C.).

In step 3) above, a compound of formula (2), wherein R is H, can be prepared by catalytic hydrogenating of a compound of formula (2-d) with heavy metal in a suitable alcohol solvent (such as methanol) and at a suitable temperature and suitable pressure (such as 0.1 Mpa). When R is hydroxyl protecting group, it is further comprising hydroxyl protecting process with the method known to the person skilled in the art following the catalytic hydrogenation step.

In a further aspect, the compound of formula (2) can reacts with organic acid or inorganic acid to obtain salts of the compound of formula (2).

Another object of the present invention is to provide a use of a compound of formula (2) or salts thereof for the preparation of triazolo pyrimidine compound. Preferably said triazolo pyrimidine compound is Ticagrelor.

A compound of formula (2),

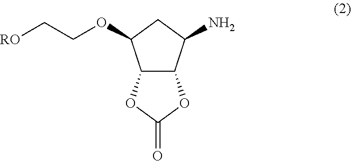

wherein, said R or salts are defined as above.

Another object of the present invention is to provide an intermediate compound of formula (1-b), (1-c), (1-d), or (1-e):

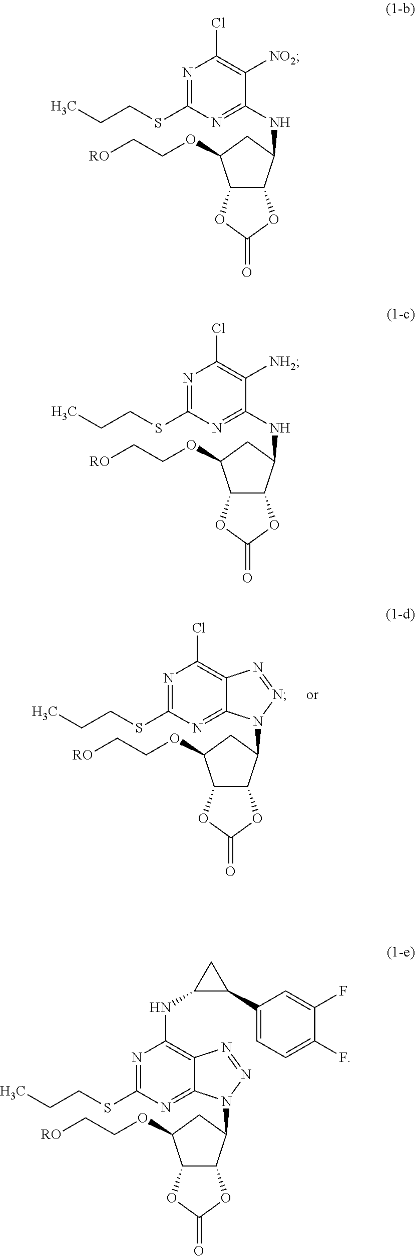

A compound of formula (2) provided by the present invention is easy to solidify. This invention provides a simple synthetic rout of a compound of formula (2) with higher purity and increased yield, which is suitable for industrial production. It is known that the intermediates used to preparing Ticagrelor in known method are all existed as oil which is not easy to purify. Surprisingly, when use a compound of formula (2) to prepare Ticagrelor, the intermediate compounds of formula (1-b), (1-c), (1-d), (1-e) obtained in the process are all in solid which is much easier to purify. The method for preparation of triazolo pyrimidine compound, particularly for preparation of Ticagrelor provided by this invention is much easier and the post-processing is greater simplicity. This method can improve the quality and yield of Ticagrelor, reduce the production cost and especially be suitable for industry production

### EXAMPLES

The invention is further illustrated by the following examples. It is to be understood that these examples are only intended to illustrate the invention, but not limit the scope of the invention. For experimental methods in the following examples without particular conditions, they are performed under routine conditions or as instructed by the manufacture. All materials, reagents are commercial available. Unless otherwise specified, all percentages, ratios, proportions or parts are by weight.

### Example 1

### Preparation of Compound (2-b′)

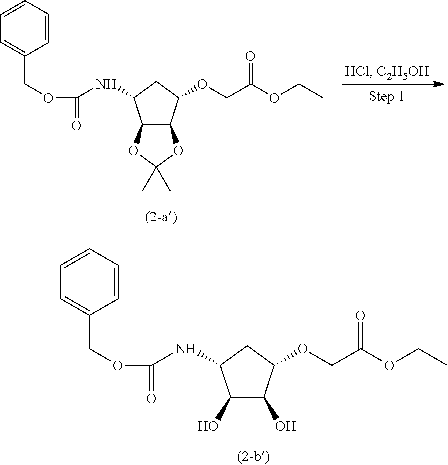 [SMILES: CCOC(=O)CO[C@H]1C[C@@H](NC(=O)OCc2ccccc2)[C@@H]2OC(C)(C)O[C@@H]21 ; CCOC(=O)CO[C@H]1C[C@@H](NC(=O)OCc2ccccc2)[C@H](O)[C@@H]1O]

160 ml ethanol, 36˜38% concentrated hydrochloric acid (60 ml, 0.70 mol) and compound (2-a′) were added a 250 ml flask to form a mixture. The reaction mixture was stirred for overnight at room temperature. The reaction was monitored by TLC. After completion, 40% aqueous sodium hydroxide was added to the mixture under the ice bath, adjusting pH of the reaction mixture to 6˜7. The ethanol was distilled off under reduced pressure. The obtained residue was extracted with ethyl acetate (200 ml*3). The organic phase was washed with saturated brine, dried with anhydrous sodium sulfate, then filtered and the ethyl acetate was removed by distillation at reduced pressure to obtain pale yellow oil (14 g, yield 77.8%), HPLC purification is above 98%.

¹H NMR (400M, CDCl₃) δ: 7.30-7.34 (m, 5H), 5.4 (s, 2H), 4.22 (s, 2H), 4.12 (q, J=13.6 Hz, 2H), 4.06-4.08 (m, 1H), 4.03-4.05 (m, 1H), 3.91-3.94 (m, 1H), 3.84-3.89 (m, 1H), 2.53-2.60 (m, 1H), 1.50-1.56 (m, 1H), 1.23-1.28 (t, J=7.6 Hz, 3H). MS (m/z): [M+H]⁺=354.10.

### Example 2

### Preparation of compound (2-c′)

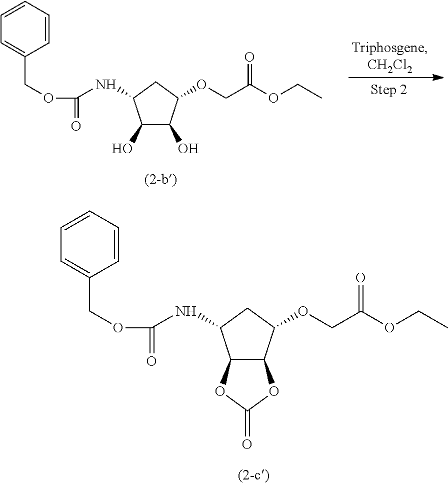 [SMILES: CCOC(=O)CO[C@H]1C[C@@H](NC(=O)OCc2ccccc2)[C@H](O)[C@@H]1O ; CCOC(=O)CO[C@H]1C[C@@H](NC(=O)OCc2ccccc2)[C@@H]2OC(=O)O[C@@H]21]

Compound (2-b′) (4 g, 11.3 mmol) and pyridine (5.5 g, 68.9 mmol) were dissolved in dichloromethane (20 ml) to form a solution. The solution was cooled to −80° C. under the nitrogen atmosphere. A solution of trichloromethane (2 g, 6.7 mmol) in dichloromethane (20 ml) was slowly added to above reaction mixture and the temperature was controlled at −80° C. during addition. After finished addition, the reaction mixture was stirred for 30 minutes, then slowly heated to room temperature and continued to stir for 30˜60 minutes. The reaction was monitored by TLC, and then quenched by aqueous ammonium chloride until there was no bubble produced in the reaction mixture. The aqueous layer was separated and the organic layer was washed with 1 mol/L hydrochloric acid, then washed with water and saturated aqueous sodium bicarbonate until the pH of the organic layer is 8˜9. The organic layer was dried with anhydrous sodium sulfate, then filtered and the organic layer was concentrated to obtain crude product (3.8 g). The crude product was isolated by column chromatography on silica (petroleum ether:ethyl acetate 2:1 as eluent). Then thus obtained yellow oil (2.4 g, yield=56.0%), HPLC purification is above 98%.

¹H NMR (400M, CDCl₃) δ: 7.31-7.36 (m, 5H), 5.10 (s, 2H), 5.06 (d, J=5.6 Hz, 1H), 5.02 (d, J=6.4 Hz, 1H), 4.26 (s, 2H), 4.21 (d, J=6.4 Hz, 1H), 4.14 (q, J=14.0 Hz, 2H), 4.08 (d, J=5.6 Hz, 1H), 2.14-2.16 (m, 1H), 2.05-2.09 (m, 1H), 1.23-1.25 (t, J=7.2 Hz, 3H). MS (m/z): [M+H]⁺=380.15, [M−H]⁻=378.00.

### Example 3

### Preparation of compound (2-d′)

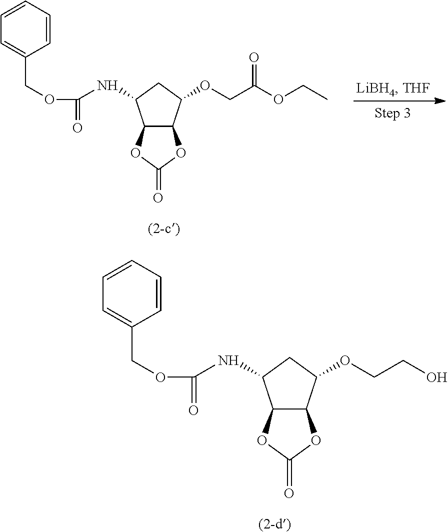 [SMILES: CCOC(=O)CO[C@H]1C[C@@H](NC(=O)OCc2ccccc2)[C@@H]2OC(=O)O[C@@H]21 ; O=C(N[C@@H]1C[C@H](OCCO)[C@H]2OC(=O)O[C@H]21)OCc1ccccc1]

A solution of 2 mol/L LiBH₄ in THF (1.32 ml, 2.64 mmol) was added to a 1000 ml flask and the temperature was cooled to −10° C. A solution of compound (2-c′) (2.64 mmol) in THF (5 ml) was slowly added to the flask above. The reaction mixture was stirred for 2 hours. The reaction was monitored by TLC. After completion, the mixture was poured into ice-water and stirred for about 15 minutes. The product was extracted by ethyl acetate (10 ml*2). The organic layer was dried with anhydrous sodium sulfate, then filtered and the ethyl acetate was removed by distillation at reduced pressure to obtain crude product (compound 2-c′) (0.8 g). The crude product was purified by column chromatography on silica (petroleum ether:ethyl acetate 3:2 as eluent). Then thus obtained pale yellow oil (0.6 g, yield=67.5%), HPLC purification is above 98%.

¹H NMR (400M, CDCl₃) δ: 7.34-7.38 (m, 5H), 5.12 (s, 2H), 5.07 (d, J=6.4 Hz, 1H), 5.03 (d, J=6.4 Hz, 1H), 4.13 (d, J=6.8 Hz, 1H), 4.09 (d, J=7.2 Hz, 1H), 3.72-3.75 (m, 2H), 3.59-3.65 (t, J=10.0 Hz, 2H), 2.10-2.12 (m, 1H), 1.97-2.04 (m, 1H). MS (m/z): [M−H]⁻=336.05.

### Example 4

### Preparation of Compound (2′)

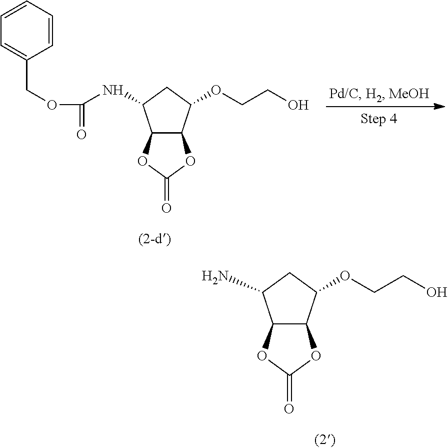 [SMILES: O=C(N[C@@H]1C[C@H](OCCO)[C@H]2OC(=O)O[C@H]21)OCc1ccccc1 ; N[C@@H]1C[C@H](OCCO)[C@H]2OC(=O)O[C@H]21]

Under an atmosphere of hydrogen (0.1 MPa), a suspension of compound (2-d′) (0.2 g, 0.6 mmol) and 10% Pd/C (0.02 g) in methanol (10 ml) was stirred for 2 hours. The reaction was monitored by TLC. After filtering, the resulting filtrate was concentrated at reduced pressure to obtain crude product (compound 2′) as a white solid (0.10 g, yield=83.3%), HPLC purification is above 99%.

¹H NMR (400M, CDCl₃) δ: 5.08 (d, J=6.8 Hz, 1H), 4.85 (d, J=6.8 Hz, 1H), 3.73-3.74 (m, 2H), 3.69-3.72 (t, J=10.8 Hz, 2H), 3.66 (d, J=6.8 Hz, 1H), 3.62 (d, J=6.4 Hz, 1H), 2.11-2.13 (m, 1H), 1.97-2.10 (m, 1H). MS (m/z): [M+H]⁺=204.20.

### Example 5

### Preparation of Hydrochloric Salt of Compound (2′)

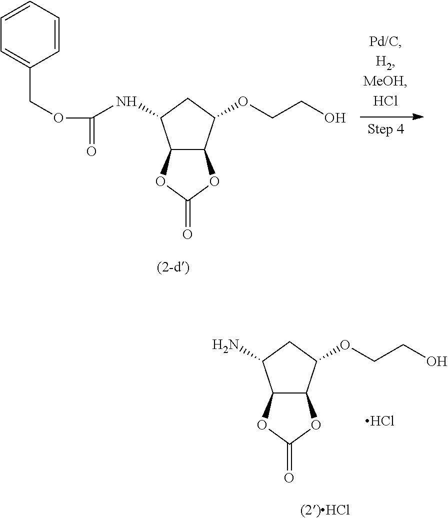 [SMILES: O=C(N[C@@H]1C[C@H](OCCO)[C@H]2OC(=O)O[C@H]21)OCc1ccccc1 ; N[C@@H]1C[C@H](OCCO)[C@H]2OC(=O)O[C@H]21 ; Cl]

Under an atmosphere of hydrogen (0.1 MPa), a suspension of compound (2-d′) (0.2 g, 0.6 mmol), concentrated hydrochloric acid (0.5 ml) and 10% Pd/C (0.02 g) in methanol (10 ml) were stirred for 2 hours. The reaction was monitored by TLC. After completion, the reaction mixture was filtered; the resulting filtrate was removed by distillation to obtain the crude product. Then the crude product was dissolved in ethyl acetate and the organic layer was washed with water, and then dried with anhydrous sodium sulfate. After filtering, the ethyl acetate was distilled off at reduced pressure to obtain the hydrochloric salt of compound 2′ as a white solid (0.19 g, yield=85.2%), HPLC purification is above 99%.

¹H NMR (400M, CDCl₃) δ: 5.09 (d, J=6.8 Hz, 1H), 4.84 (d, J=6.8 Hz, 1H), 3.72-3.74 (m, 2H), 3.68-3.71 (t, J=10.8 Hz, 2H), 3.66 (d, J=6.8 Hz, 1H), 3.62 (d, J=6.4 Hz, 1H), 2.10-2.12 (m, 1H), 1.96-2.10 (m, 1H). MS (m/z): [M+H]⁺=204.20.

### Example 6

### Preparation of Acetic Salt of Compound (2′)

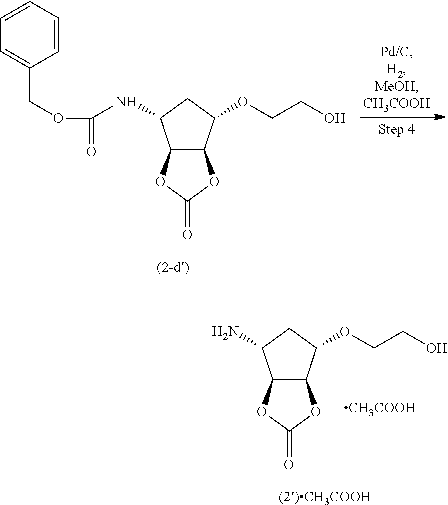

Under an atmosphere of hydrogen (0.1 MPa), a suspension of compound (2-d′) (0.2 g, 0.6 mmol), acetic acid (0.036 g, 0.6 mmol) and 10% Pd/C (0.02 g) in methanol (10 ml) were stirred for 2 hours. The reaction was monitored by TLC. After completion, the reaction mixture was filtered; the resulting filtrate was removed by distillation at reduced pressure to obtain the acetic salt of compound 2′ as a white solid (0.20 g, yield=86.8%), HPLC purification is above 99%.

¹HNMR (400M, MeOD) δ: 5.08 (d, J=6.8 Hz, 1H), 4.85 (d, J=6.8 Hz, 1H), 3.73-3.74 (m, 2H), 3.69-3.72 (t, J=10.8 Hz, 2H), 3.66 (d, J=6.8 Hz, 1H), 3.62 (d, J=6.4 Hz, 1H), 2.25 (s, 3H), 2.11-2.13 (m, 1H), 1.97-2.10 (m, 1H). MS (m/z): [M+H]⁺=204.20.

### Example 7

### Preparation of L-tartaric salt of compound (2′)

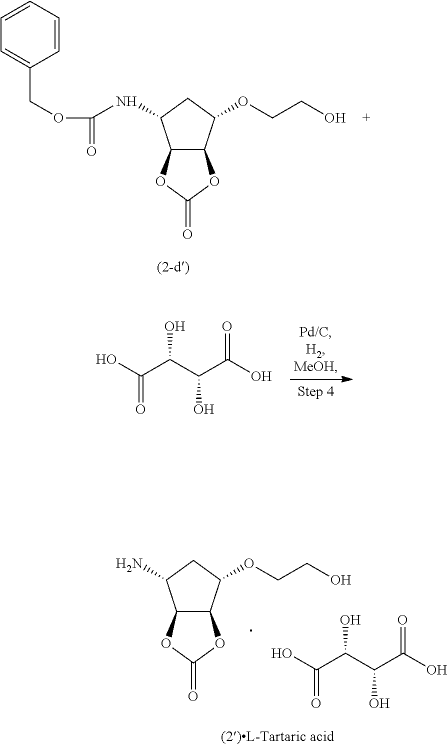

Under an atmosphere of hydrogen (0.1 MPa), a suspension of compound (2-d′) (0.2 g, 0.6 mmol), L-tartaric acid (0.09 g, 0.6 mmol) and 10% Pd/C (0.02 g) in methanol (10 ml) were stirred for 2 hours. The reaction was monitored by TLC. After completion, the reaction mixture was filtered; the resulting filtrate was removed by distillation at reduced pressure to obtain the L-tartaric salt of compound 2′ as a white solid (0.24 g, yield=83.1%), HPLC purification is above 99%.

¹HNMR (400M, MeOD) δ: 5.08 (d, J=6.8 Hz, 1H), 4.83 (d, J=6.8 Hz, 1H), 4.4 (s, 2H), 3.72-3.74 (m, 2H), 3.68-3.71 (t, J=10.8 Hz, 2H), 3.66 (d, J=6.8 Hz, 1H), 3.62 (d, J=6.4 Hz, 1H), 2.11-2.13 (m, 1H), 1.97-2.10 (m, 1H). MS (m/z): [M+H]⁺=204.20.

### Example 8

### Preparation of Compound (2″) (R=Bn)

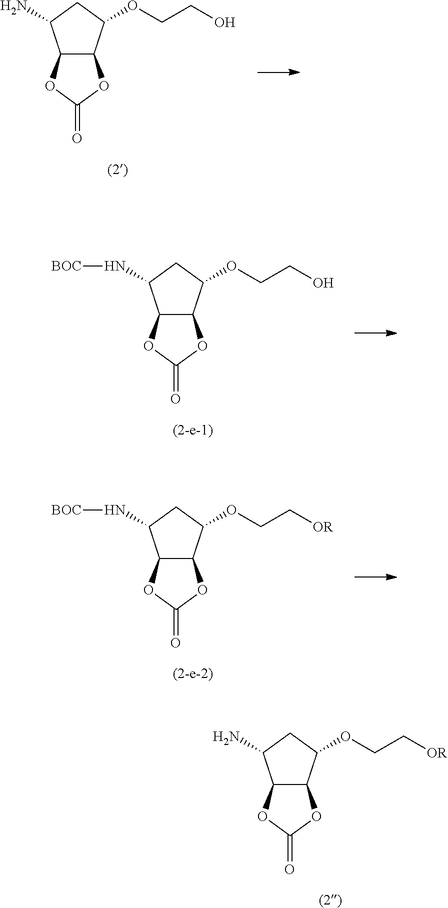

Preparation of compound (2-e-1): Compound (2′) (1.05 g, 5.18 mmol) was dissolved in anhydrous dichloromethane (10 ml). Triethylamine (0.72 ml, 5.18 mmol), di-tert-butyl dicarbonate (abbreviated (BOC)₂O)) (2.26 g, 10.4 mmol) and 4-dimethylaminopyridine (abbreviated DMAP) (0.63 g, 5.18 mmol) were added to the solution. The resulting mixture was stirred at room temperature for 6˜10 hours under the nitrogen atmosphere, followed by addition of dichloromethane (40 ml). Then the organic layer was washed with citric acid (20 ml*2). The organic layer was removed by distillation at reduced pressure to obtain white solid. The white solid was used directly in the next step.

MS (m/z) [M+H]⁺=304.20.

Preparation of compound (2-e-2): compound (2-e-1) (1.5 g, about 5 mmol) was dissolved in anhydrous dichloromethane (18 ml). Ag₂O (2.34 g, 10.1 mmol) and 4 Å molecular sieve (1.8 g) were added to the solution in sequence. Under the nitrogen atmosphere; BnBr (720 μl, 6.06 mmol) was added to the reaction mixture at 0° C. Then the resulting mixture was heated to room temperature (25˜30° C.) and maintained for 2˜3 days. After completion, the mixture was filtered through celatom. The resulting filtrate was concentrated at reduced pressure and purified by column chromatography on silica (petroleum ether:ethyl acetate 3:2 as eluent). Then thus obtained white solid (1.3 g, yield=62.5%), MS (m/z): [M+H]⁺=394.32.

Preparation of compound (2″) (R=Bn): Compound (2-e-2) (1.3 g, 3.3 mmol) was dissolved in dichloromethane (10 ml) and the solution was stirred for overnight at room temperature. The reaction was monitored by TLC. After completion, dichloromethane was distilled off leaving a residue, followed by addition of cyclohexane (10 ml*2) and then the cyclohexane was distilled off to remove the unreacted trifluoroacetic acid. The resulting solid was dissolved in dichloromethane (10 ml) and 10% aqueous sodium bicarbonate (3 g, 3.3 mmol) was added slowly to the solution at −5˜0° C. and stirred for 15-10 min. The aqueous and organic layers were separated and the organic layer was dried with anhydrous sodium sulfate. After filtering, the dichloromethane was removed by distillation at reduced pressure to obtain the white solid (0.88 g, yield=90%), HPLC purification is 99%.

1H NMR (400M, CDCl₃) δ: 7.32-7.36 (m, 5H), 5.38 (s, 2H), 5.10 (d, J=6.4 Hz, 1H), 4.84 (d, J=6.4 Hz, 1H), 3.85-3.90 (m, 4H), 3.65 (d, J=6.4 Hz, 1H), 3.63 (d, J=6.8 Hz, 1H), 2.10-2.14 (m, 1H), 1.95-2.09 (m, 1H). MS (m/z): [M+H]+=294.13.

### Example 9

### Preparation of Hydrochloric Salt of Compound (2″) (R=Bn)

The solution of compound (2″) (5.9 g, 20 mmol) in ethyl acetate (30 ml) was slowly added the solution of concentrated hydrochloric acid (1.67 ml) in methanol (10 ml) while stirring. White solid was precipitated during the addition. After the addition finished, the reaction mixture was filtered and the resulting solid was washed with ethyl acetate and then dried in vacou to obtain a white solid (hydrochloric salt of compound (2″) (R=Bn)) (5.9 g, yield=89.0%), HPLC purification is 99%.

¹HNMR (400M, CDCl₃) δ: 7.32-7.36 (m, 5H), 5.38 (s, 2H), 5.10 (d, J=6.4 Hz, 1H), 4.84 (d, J=6.4 Hz, 1H), 3.85-3.90 (m, 4H), 3.65 (d, J=6.4 Hz, 1H), 3.63 (d, J=6.8 Hz, 1H), 2.10-2.14 (m, 1H), 1.95-2.09 (m, 1H). MS (m/z): [M+H]⁺=294.13.

### Example 10

### Preparation of Acetic Salt of Compound (2″) (R=Bn)

Compound (2″) (R=Bn) (5.9 g, 20 mmol) was dissolved in ethyl acetate (30 ml) and acetic acid (1.2 g, 20 mmol) was dissolved in methanol (10 ml). The solution of compound (2″) (R=Bn) in ethyl acetate was slowly added to the solution of acetic acid in methanol while stirring. White solid was precipitated during the addition. After the addition finished, the reaction mixture was filtered and the resulting solid was washed with ethyl acetate and then dried in vacou to obtain a white solid (acetic salt of compound (2″) (R=Bn))(6.5 g, yield=92.0%), HPLC purification is 99%.

¹H NMR (400M, MeOD) δ: 7.33-7.36 (m, 5H), 5.37 (s, 2H), 5.10 (d, J=6.4 Hz, 1H), 4.83 (d, J=6.4 Hz, 1H), 3.84-3.90 (m, 4H), 3.64 (d, J=6.4 Hz, 1H), 3.62 (d, J=6.8 Hz, 1H), 2.26 (s, 3H), 2.11-2.14 (m, 1H), 1.95-2.10 (m, 1H). MS (m/z): [M+H]⁺=294.13.

### Example 11

### Preparation of L-Tartaric Salt of Compound (2″) (R=Bn)

Compound (2″) (R=Bn) (5.9 g, 20 mmol) was dissolved in ethyl acetate (30 ml) and L-tartaric acid (3.0 g) was dissolved in methanol (10 ml). The solution of compound (2″) (R=Bn) in ethyl acetate was slowly added to the solution of L-tartaric acid in methanol while stirring. White solid was precipitated during the addition. After the addition finished, the reaction mixture was filtered and the resulting solid was washed with ethyl acetate and then dried in vacou to obtain a white solid (L-tartaric salt of compound (2″) (R=Bn)) (7.8 g, yield=88.2%), HPLC purification is 99%.

¹HNMR (400M, MeOD) δ: 7.32-7.35 (m, 5H), 5.38 (s, 2H), 5.11 (d, J=6.4 Hz, 1H), 4.82 (d, J=6.4 Hz, 1H), 4.5 (s, 2H), 3.85-3.91 (m, 4H), 3.66 (d, J=6.4 Hz, 1H), 3.64 (d, J=6.8 Hz, 1H), 2.11-2.14 (m, 1H), 1.96-2.10 (m, 1H). MS (m/z): [M+H]⁺=294.13.

### Example 12

### Preparation of Compound (1-b′)

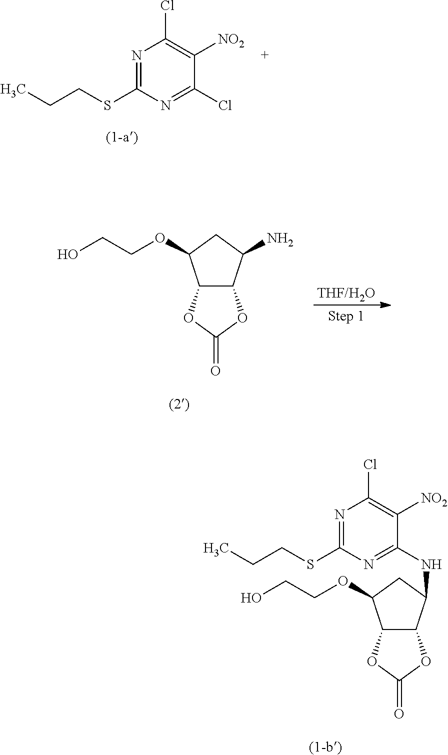

Compound (1-a′) (402 mg, 1.5 mmol) and compound (2′) (335 mg, 1.65 mmol) were dissolved in THF (15 ml) and the solution was stirred for 2˜3 hours at 0˜10° C., followed by addition of water (20 ml). The resulting mixture was extracted by ethyl acetate (15 ml*3). The organic layer was dried by anhydrous sodium sulfate, then filtered and the resulting filtrate was removed by distillation to obtain the crude product as a pale yellow solid. The crude product was crystallized from a mixture of ethyl acetate and petroleum ether to obtain a pale yellow solid (332 mg, yield 51.0%), HPLC purification is above 98%.

¹H NMR (400M, CDCl₃) δ: 8.60 (b, 1H), 4.63-4.74 (m, 2H), 4.50-4.56 (m, 1H), 3.97 (d, J=7.6 Hz, 1H), 3.68-3.84 (m, 3H), 3.63-3.66 (m, 1H), 3.04-3.21 (m, 2H), 2.30-2.34 (m, 1H), 1.92-1.95 (m, 1H), 1.74-1.81 (m, 2H), 1.09 (t, J=7.6 Hz, 3H). MS (m/z): [M+H]⁺=435.20.

### Example 13

### Preparation of Compound (1-c′)

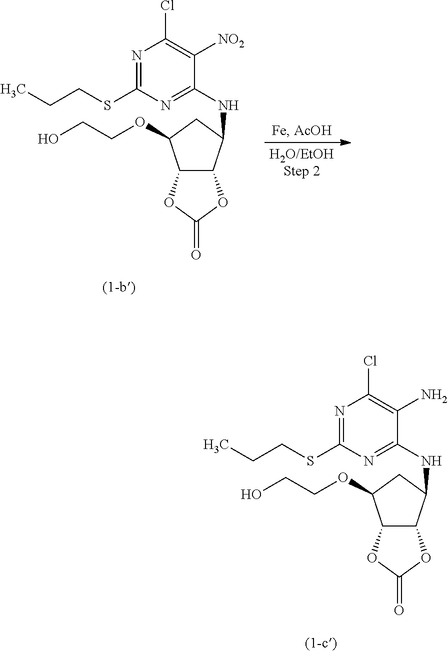

Compound (1-b′) (1.5 g, 3.5 mmol), iron power (2.0 g, 35 mmol) and glacial acetic acid (2.1 g, 35 mmol) were dissolved in the mixture of water/ethanol (20 ml). Under the nitrogen atmosphere, the reaction mixture was stirred for 30˜60 min at 60˜70° C. The reaction was monitored by TLC. After completion, the mixture was filtered and the resulting filtrate was extracted with dichloromethane (20 ml*3). The organic layer was dried with anhydrous sodium sulfate and then filtered. The dichloromethane was removed by distillation at reduced pressure to obtain crude product (compound (1-c′)) and the crude product was used for the next step directly without further purification.

MS (m/z): [M+H]⁺=405.87.

### Example 14

### Preparation of Compound Formula (1-c′)

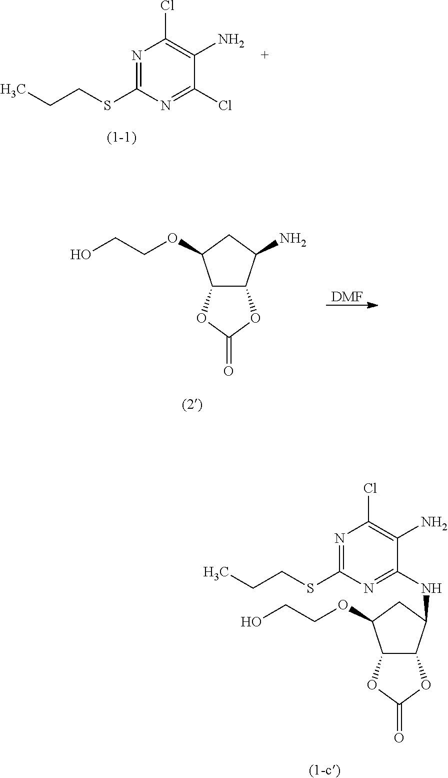

Compound (1-1) (476 mg, 2.0 mmol), compound (2′) (446 mg, 2.2 mmol) and NaHCO₃ (185 mg, 2.2 mmol) were added to DMF (20 ml). Under the nitrogen atmosphere, the reaction mixture was stirred for 12˜15 hours at 90˜95° C. The reaction was monitored by TLC. After completion, water (100 ml) and CH₂Cl₂ (100 ml) were added to the reaction mixture. The resulting mixture was stirred for 30 min, followed by the separation of the aqueous and organic layers. The dichloromethane layer was washed with water (20 ml*3) until to neutral. The resulting organic layer was washed with anhydrous sodium sulfate, and then filtered. The filtrate was removed by distillation at reduced pressure to obtain crude product (compound (1-c′)). The crude product was crystallized from the mixture of dichloromethane and petroleum ether to obtain a white solid (680 mg, yield 85.0%). HPLC purification is above 98%.

¹HNMR (400 MHz, CDCl₃) δ: 6.80 (b, 2H), 4.62-4.72 (m, 2H), 4.52-4.54 (m, 1H), 3.95 (d, J=7.6 Hz, 1H), 3.66-3.82 (m, 3H), 3.63-3.64 (m, 1H), 3.03-3.22 (m, 2H), 2.30-2.32 (m, 1H), 1.92-1.94 (m, 1H), 1.73-1.82 (m, 2H), 1.08 (t, J=7.6 Hz, 3H). MS (m/z): [M+H]⁺=405.86.

### Example 15

### Preparation of Compound (1-c′) from the Hydrochloric Salt of Compound (2′)

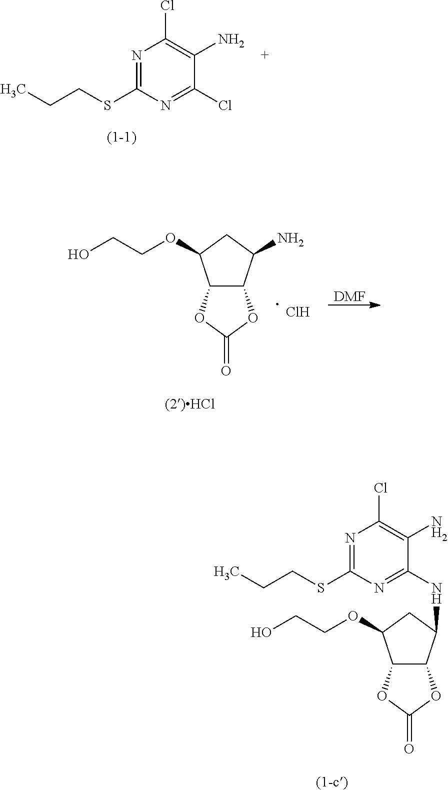

Compound (1-1) (476 mg, 2.0 mmol), hydrochloric salt of compound (2′) (524 mg, 2.2 mmol) and NaHCO₃ (370 mg, 4.4 mmol) were added to DMF (20 ml). Under the nitrogen atmosphere, the reaction mixture was stirred for 12˜15 hours at 90˜95 [symbol] . The reaction was monitored by TLC. After completion, water (100 ml) and CH₂Cl₂ (100 ml) were added to the reaction mixture. The resulting mixture was stirred for 30 min, followed by the separation of the aqueous and organic layers. The dichloromethane layer was washed with water (20 ml*3) until to neutral. The resulting organic layer was dried with anhydrous sodium sulfate, and then filtered. The filtrate was removed by distillation at reduced pressure to obtain crude product (compound (1-c′)). The crude product was crystallized from the mixture of dichloromethane and petroleum ether to obtain a white solid (656 mg, yield 82.0%). HPLC purification is above 98%.

¹HNMR (400 MHz, CDCl₃) δ: 6.81 (b, 2H), 4.62-4.72 (m, 2H), 4.51-4.53 (m, 1H), 3.95 (d, J=7.6 Hz, 1H), 3.67-3.82 (m, 3H), 3.63-3.64 (m, 1H), 3.03-3.22 (m, 2H), 2.30-2.33 (m, 1H), 1.92-1.95 (m, 1H), 1.73-1.82 (m, 2H), 1.09 (t, J=7.6 Hz, 3H), MS (m/z): [M+H]⁺=405.86.

Preparation of compound (1-c′) from the acetic salt or tartaric salt of compound (2′) was similar to that described in example 15.

### Example 16

### Preparation of Compound of (1-d′)

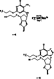 [SMILES: CCCSc1nc(Cl)c(N)c(N[C@@H]2C[C@H](OCCO)[C@H]3OC(=O)O[C@H]32)n1 ; "NaNO2" ; "H2O, AcOH, PhMeStep 3" ; CCCSc1nc(Cl)c2nnn([C@@H]3C[C@H](OCCO)[C@H]4OC(=O)O[C@H]43)c2n1]

To a solution of compound (1-c′9) (about 1.4 g, 3.5 mmol) and glacial acetic acid (1.3 g, 21 mmol) in toluene (20 ml) was added a solution of sodium nitrite (0.27 g, 4.0 mmol) in water. The resulting mixture was stirred for 30˜60 min at 20˜30° C. Then the pH of the reaction mixture was adjusted to 8˜9 with saturate sodium bicarbonate, and then filtered. The organic layer was extracted with ethyl acetate (20 ml*3). The combined ethyl acetate layer was dried with anhydrous sodium sulfate, and then filtered. The filtrate was distilled off under the reduced pressure. The residue was purified with column chromatography on silica (petroleum ether:ethyl acetate 15:1 as eluent) to obtain a white solid (compound (1-d′) (0.77 g, yield: 55.2%), HPLC purification is above 98%.

¹H NMR (400M, CDCl₃) δ: 5.54 (q, 1H), 5.22-5.24 (m, 1H), 4.88 (d, J=6.8 Hz, 1H), 4.03-4.08 (m, 1H), 3.48-3.64 (m, 4H), 3.23 (t, J=7.6 Hz, 2H), 2.66-2.70 (m, 1H), 2.54-2.57 (m, 1H), 1.80-1.88 (m, 2H), 1.10 (t, J=7.6 Hz, 3H). MS (m/z): [M+H]⁺=416.59.

### Example 17

### Preparation of Compound (1-e′)

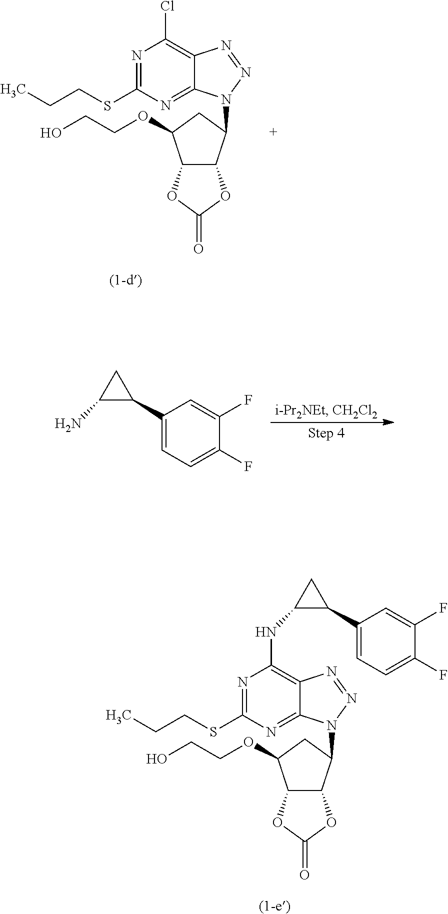

Compound (1-d′) (830 mg, 2.0 mmol) and (1R,2S)-2-(3,4-difluorophenyl)cyclopropanamine (338 mg, 2.5 mmol) were dissolved in dichloromethane (15 ml). The reaction mixture was stirred for 15˜20 hours at 20˜30° C. The reaction was monitored by TLC. After completion, to the reaction mixture was added water (15 ml) and the mixture was stirred for 10˜25 min, followed by the separated of aqueous and organic layers. The aqueous layer was extracted with dichloromethane (15 ml*2). The combined organic layer was dried with anhydrous sodium sulfate, and then filtered. The resulting filtrate was distilled under the reduced pressure to obtain the crude product. The crude product was crystallized from the mixture of ethyl acetate and petroleum ether to provide a white solid (1.04 g, yield: 98%), HPLC purification is above 98%.

¹H NMR (400M, CDCl₃) δ: 7.06-7.13 (m, 3H), 5.99 (q, 1H), 5.28-5.35 (m, 1H), 4.24-4.25 (m, 1H), 4.22-4.23 (m, 1H), 3.55-3.78 (m, 4H), 3.07-3.10 (m, 2H), 3.03-3.05 (m, 1H), 2.98-2.99 (m, 1H), 2.16-2.32 (m, 1H), 2.14-2.15 (m, 1H), 1.68-1.74 (m, 2H), 1.64-1.66 (m, 2H), 0.95 (t, J=14.8 Hz 3H). MS (m/z): [M+H]⁺=549.00, [M−H]⁻=547.00.

### Example 18

### Preparation of compound 1 (Ticagrelor)

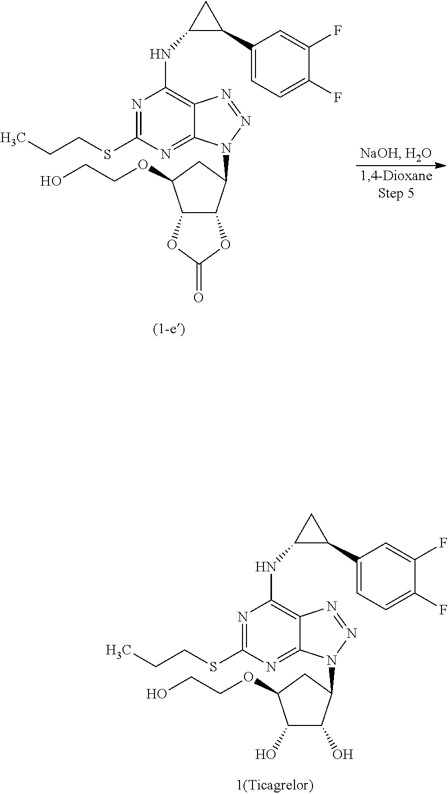

A solution of sodium hydroxide (0.88 g, 22 mmol) in water (20 mml) was added slowly to a solution of compound (1-e′) (0.6 g, 1.1 mmol) in 1,4-dioxane (15 ml) cooling the temperature during the addition with an ice bath. After addition, the temperature of the reaction mixture was slowly heated to 20˜30° C. and stirring for 2˜3 hours. The reaction was monitored with TLC. After completion, aqueous ammonium chloride was added to adjust the pH of the mixture to 7˜8. The organic layer was extracted by ethyl acetate (20 ml*3). Then the combined ethyl acetate was dried with anhydrous sodium sulfate, then filtered and the ethyl acetate was removed by distillation at reduced pressure to obtain crude product. The crude product was purified with column chromatography on silica (dichloromethane:methanol 100:1˜50:1 gradient elution) to obtain compound 1 (Ticagrelor) as a white solid (0.48 g, yield 85.2%). HPLC purification is above 99%.

¹H NMR (400M, MEOD) δ: 7.06-7.23 (m, 3H), 5.12 (q, 1H), 4.73-4.77 (m, 1H), 4.16-4.18 (m, 1H), 3.89-3.93 (m, 1H), 3.60-3.71 (m, 4H), 3.04-3.31 (m, 2H), 2.89-2.93 (m, 1H), 2.73-2.80 (m, 1H), 2.19-2.26 (m, 1H), 2.10-2.18 (m, 1H), 1.58-1.64 (m, 2H), 1.45-1.48 (m, 1H), 1.36-1.39 (m, 1H), 0.93 (t, J=14.8 Hz, 3H). MS (m/z): [M+H]⁺=523.00.

### Example 19

### Preparation of Compound (1-c″) (R=Bn)

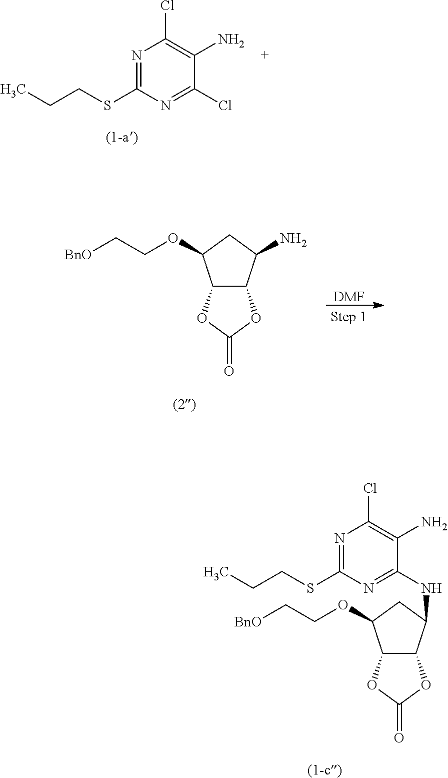

Compound (1-a′) (524 mg, 2.2 mmol), compound (2″) (703 mmg, 2.4 mmol) and NaHCO₃ (203 mg, 2.4 mmol) were added to DMF (25 ml). Under the nitrogen atmosphere, the mixture was stirred for 1215 h at 9095° C. The reaction was monitored by TLC. After completion, H₂O (110 ml) and CH₂Cl₂ (110 ml) were added to the mixture and then stirring for 30 min. Then the aqueous and organic layers were separated. The CH₂Cl₂ was washed to neutral with H₂O (25 ml*3). The organic layer was dried with anhydrous sodium sulfate, and then filtered. The CH₂Cl₂ layer was removed by distillation at reduced pressure to obtain crude product (compound (1-c′). The crude product was crystallized from a mixture of dichloromethane and petroleum ether to obtain a white solid (933 mg, yield 79.0%). HPLC purification is above 98%.

¹HNMR (400 MHz, CDCl₃) δ: 7.31-7.34 (m, 5H), 6.79 (b, 2H), 5.36 (s, 2H), 4.60-4.72 (m, 2H), 4.51-4.53 (m, 1H), 3.94 (d, J=7.6 Hz, 1H), 3.64-3.80 (m, 3H), 3.63-3.64 (m, 1H), 3.02-3.22 (m, 2H), 2.31-2.33 (m, 1H), 1.93-1.94 (m, 1H), 1.72-1.82 (m, 2H), 1.08 (t, J=7.6 Hz, 3H). MS (m/z): [M+H]⁺=495.15.

### Example 20

### Preparation of compound (1-c″) from hydrochloric salt of compound 2″(R=Bn)

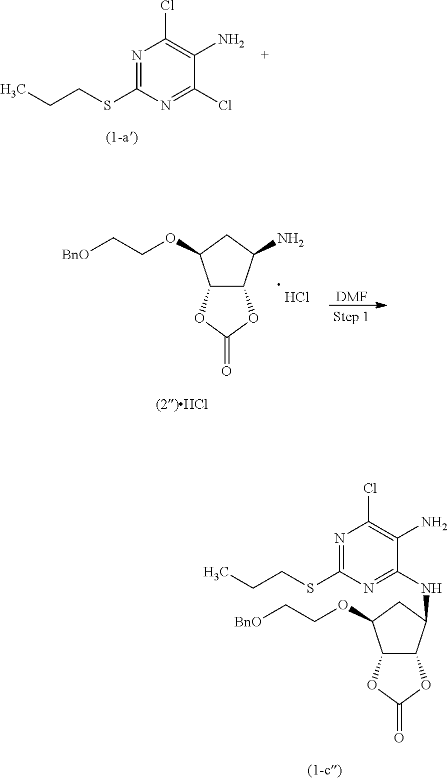

Compound (1-a′) (524 mg, 2.2 mmol), hydrochloric salt of compound 2″ (793 mg, 2.4 mmol) and NaHCO₃ (406 mg, 4.81=01) were added to DMF (25 ml). Under the nitrogen atmosphere, the mixture was stirred for 12˜15 h at 90˜95° C. The reaction was monitored by TLC. After completion, H₂O (110 ml) and CH₂Cl₂ (110 ml) were added to the mixture and then stirring for 30 min. Then the aqueous and organic layers were separated. The CH₂Cl₂ layer was washed to neutral with H₂O (25 ml*3). The organic layer was dried with anhydrous sodium sulfate, and then filtered. The CH₂Cl₂ was removed by distillation at reduced pressure to obtain crude product (compound (1-c′). The crude product was crystallized from a mixture of dichloromethane and petroleum ether to obtain a white solid (886 mg, yield 75.2%). HPLC purification is above 98%.

¹HNMR (400 MHz, CDCl₃) δ: 7.32-7.35 (m, 5H), 6.79 (b, 2H), 5.36 (s, 2H), 4.61-4.72 (m, 2H), 4.51-4.53 (m, 1H), 3.95 (d, J=7.6 Hz, 1H), 3.64-3.80 (m, 3H), 3.63-3.64 (m, 1H), 3.02-3.22 (m, 2H), 2.31-2.33 (m, 1H), 1.93-1.94 (m, 1H), 1.71-1.82 (m, 2H), 1.09 (t, J=7.6 Hz, 3H). MS (m/z): [M+H]⁺=495.15.

Preparation of compound (1-c″) from acetic salt or tartaric salt of compound 2″ is similar to that described in example 18.

### Example 21

### Preparation of Compound I (Ticagrelor) from Compound (1-c″) (R=Bn)

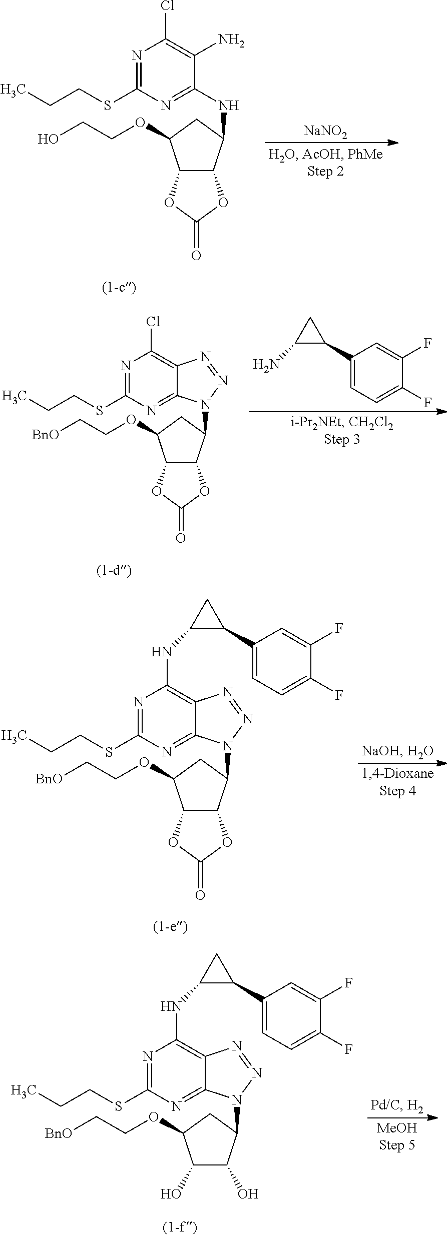

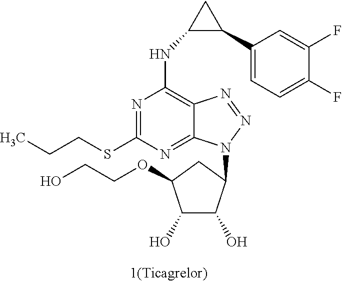 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

Preparation of compound (1-d″) from compound (1-c″): crude product of compound (1-c″) and glacial acetic acid (2.9 g, 47 mmol) were dissolved in toluene (45 ml). Sodium nitrite (0.60 g, 10.0 mmol) was dissolved in water (6 ml) and then added to the solution of glacial acetic acid in toluene above. The resulting mixture was stirred for 30˜60 min at 20˜30° C. Then aqueous saturate sodium bicarbonate was added to the mixture to adjust the pH to 8˜9 then filtered. The organic layer was extracted with ethyl acetate (45 ml*3). The combined ethyl acetate was dried with anhydrous sodium sulfate then filtered and the solution was removed by distillation at reduced pressure. The residue was purified with column on silica (petroleum ether:ethyl acetate 15:1 as eluent) to obtain compound (1-d″) as a white solid (3.41 g, yield=88.1%). HPLC purification is above 98%.

¹HNMR (400M, CDCl₃) δ: 7.32-7.35 (m, 5H), 5.53 (q, 1H), 5.39 (s, 2H), 5.21-5.23 (m, 1H), 4.87 (d, J=6.8 Hz, 1H), 4.02-4.07 (m, 1H), 3.46-3.63 (m, 4H), 3.23 (t, J=7.6 Hz, 2H), 2.65-2.71 (m, 1H), 2.53-2.57 (m, 1H), 1.79-1.88 (m, 2H), 1.09 (t, J=7.6 Hz, 3H). MS (m/z): [M+H]⁺=506.12.

Preparation of compound (1-e″) from compound (1-d″): Compound (1-d″) (1.6 g, 2.7 mmol), (1R,2S)-2-(3,4-difuorophenyl)-cyclopropylamine (912 mg, 2.7 mmol) and N,N-diisopropyl ethylamine (448 mg, 3.3 mmol) were dissolved in dichloromethane (22 ml) and the reaction mixture was stirred for 15˜20 h at room temperature (20˜30° C.). The reaction was monitored by TLC. After completion, H₂O (20 ml) was added to the mixture and then stirring for 10˜25 min. Then the aqueous and organic layers were separated. The aqueous was extracted with CH₂Cl₂ (25 ml*2). The combination CH₂Cl₂ layer was dried with anhydrous sodium sulfate, and then filtered. The CH₂Cl₂ was removed by distillation at reduced pressure to obtain crude product (compound (1-e″). The crude product was crystallized from a mixture of ethyl acetate and petroleum ether to obtain a white solid (1.58 g, yield 92.1%). HPLC purification is above 98%.

¹HNMR (400M, CDCl₃) δ: 7.32-7.36 (m, 5H), 7.05-7.13 (m, 3H), 5.97 (q, 1H), 5.37 (s, 2H), 5.27-5.37 (m, 1H), 4.24-4.26 (m, 1H), 4.21-4.23 (m, 1H), 3.54-3.78 (m, 4H), 3.07-3.11 (m, 2H), 3.02-3.06 (m, 1H), 2.96-3.01 (m, 1H), 2.15-2.31 (m, 1H), 2.13-2.15 (m, 1H), 1.67-1.74 (m, 2H), 1.63-1.65 (m, 2H), 0.97 (t, J=14.8 Hz 3H). MS (m/z): [M+H]⁺=639.23.

Preparation of compound (1-f″) from compound (1-e″): A solution of compound (1-e″) (12.8 g, 22 mmol) in 1,4-dioxane (300 ml) was added slowly to a solution of sodium hydroxide (17.6 g, 440 mmol) in water (400) cooling the temperature during the addition with an ice bath. After addition, the temperature of the reaction mixture was slowly heated to 20˜30° C. and stirred for 2˜3 hours. The reaction was monitored with TLC. After completion, aqueous ammonium chloride was added to adjust the pH of the mixture to 7˜8. The organic layer was extracted by ethyl acetate (400 ml*3). Then the combined ethyl acetate was dried with anhydrous sodium sulfate, then filtered and the ethyl acetate was removed by distillation at reduced pressure to obtain crude product. The crude product was purified with column chromatography on silica (dichloromethane:methanol 100:1˜50:1 gradient elution) to obtain compound (1-f″) as a white solid (10.2 g, yield 83.0%). HPLC purification is above 98%.

¹HNMR (400M, MeOD) δ: 7.33-7.37 (m, 5H), 7.05-7.22 (m, 3H), 5.37 (s, 2H), 5.11 (q, 1H), 4.72-4.78 (m, 1H), 4.15-4.19 (m, 1H), 3.90-3.93 (m, 1H), 3.61-3.71 (m, 4H), 3.05-3.30 (m, 2H), 2.88-2.94 (m, 1H), 2.72-2.82 (m, 1H), 2.18-2.27 (m, 1H), 2.09-2.17 (m, 1H), 1.57-1.63 (m, 2H), 1.44-1.48 (m, 1H), 1.36-1.40 (m, 1H), 0.95 (t, J=14.8 Hz, 3H). MS (m/z): [M+H]⁺=613.25.

Preparation of compound 1 (Ticagrelor) from compound (1-f″): Compound (1-f″) (11.0 g, 18 mmol) was dissolved in anhydrous methanol (300 ml) to form a mixture. 10% Pd/C (1.1 g) and methanol (200 ml) were added to the above mixture. Under a pressurized hydrogen atmosphere (0.4 MPa), a suspension of the resulting mixture was stirred for 2 hours. The reaction was monitored with TLC. After completion, the mixture was filtered. The solvent of the filtration was removed by distillation to get crud product. The crude product was crystallized from a mixture of isopropanol and n-heptane to obtain compound 1 as a white solid (8.9 g, yield 95.2%). HPLC purification is above 98%.

¹HNMR (400M, MeOD) δ: 7.06-7.23 (m, 3H), 5.11 (q, 1H), 4.72-4.76 (m, 1H), 4.15-4.18 (m, 1H), 3.89-3.92 (m, 1H), 3.60-3.72 (m, 4H), 3.05-3.31 (m, 2H), 2.89-2.94 (m, 1H), 2.72-2.80 (m, 1H), 2.18-2.26 (m, 1H), 2.11-2.18 (m, 1H), 1.58-1.65 (m, 2H), 1.46-1.48 (m, 1H), 1.36-1.40 (m, 1H), 0.95 (t, J=14.8 Hz, 3H). MS (m/z): [M+H]⁺=523.01.

1-16. (canceled)

17. A process for preparing Ticagrelor, comprising:
1) coupling a compound of formula (1-d) with a compound of formula (3) or salt thereof to provide a compound of formula (1-e): 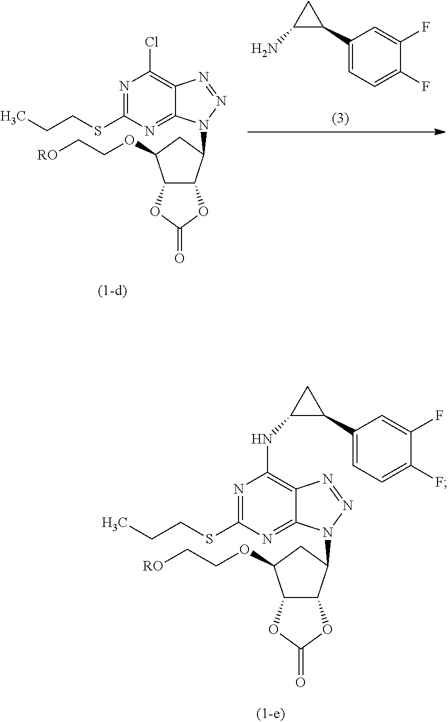
and,
2) deprotecting a compound of formula (1-e) to provide Ticagrelor: 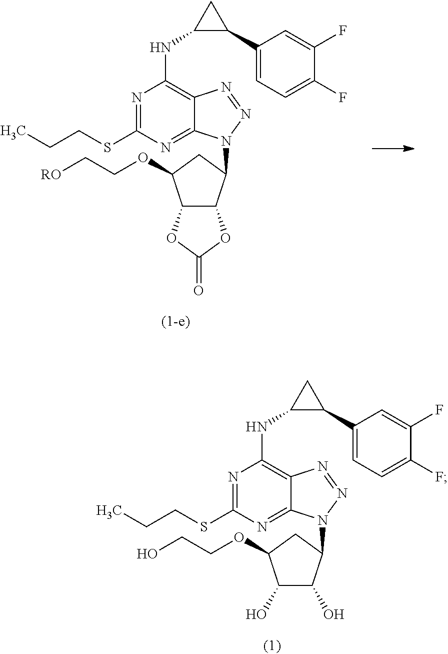
Wherein, R is H or hydroxyl protecting group.

18. A process according to claim 17, wherein a compound of formula (1-d) is prepared by cyclization of a compound of formula (1-c):

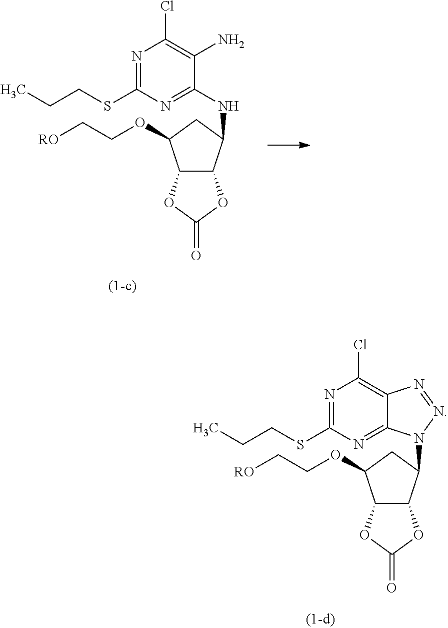

19. A process according to claim 18, wherein a compound of formula (1-c) is prepared by reducing of a compound of formula (1-b):

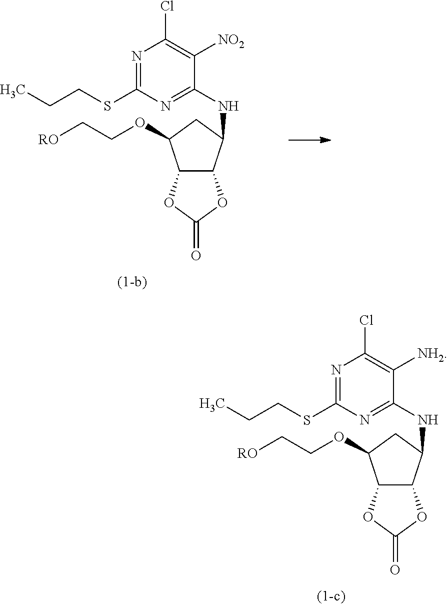

20. A process according to claim 18, wherein a compound of formula (1-c) is prepared by reacting compound of formula (1-1) with a compound of formula (2) or salts thereof:

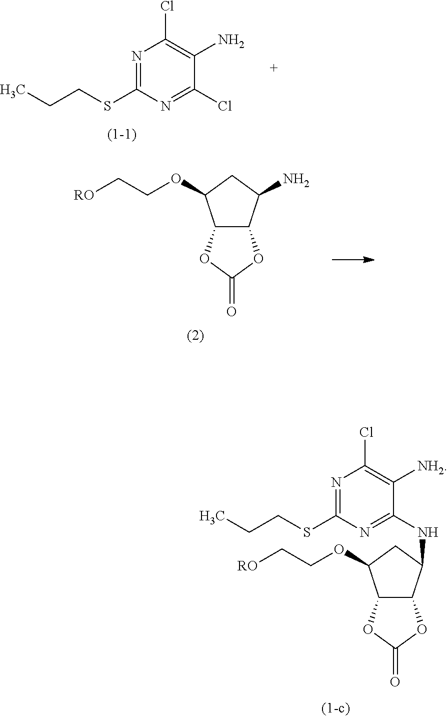

21. A process according to claim 19, wherein a compound of formula (1-b) is prepared by reacting a compound of formula (1-a) with a compound of formula (2) or salts thereof:

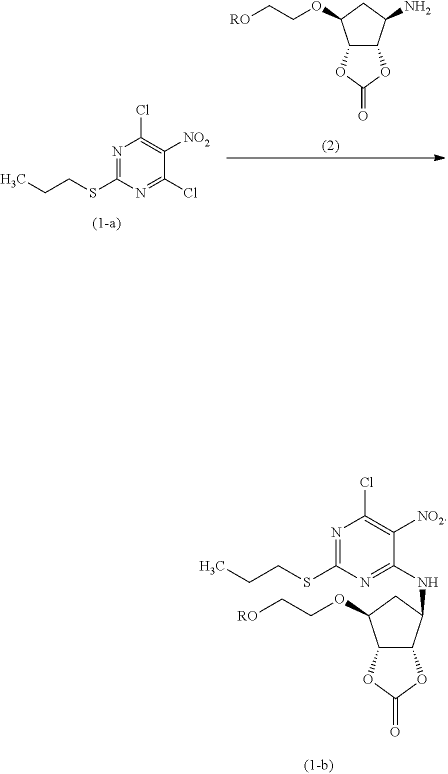

22. A process according to claim 17, wherein said hydroxyl protecting group is selected from the group consisting of silicyl, triphenylmethyl or substituted triphenylmethyl, benzyl or substituted benzyl, alkoxylmethyl or methyl substituted by alkoxyl, acyl, allyl or substituted allyl or alkoxycarbonyl.

23. A process according to claim 18, wherein said hydroxyl protecting group is selected from the group consisting of silicyl, triphenylmethyl or substituted triphenylmethyl, benzyl or substituted benzyl, alkoxylmethyl or methyl substituted by alkoxyl, acyl, allyl or substituted allyl or alkoxycarbonyl.

24. A process according to claim 19, wherein said hydroxyl protecting group is selected from the group consisting of silicyl, triphenylmethyl or substituted triphenylmethyl, benzyl or substituted benzyl, alkoxylmethyl or methyl substituted by alkoxyl, acyl, allyl or substituted allyl or alkoxycarbonyl.

25. A process according to claim 20, wherein said hydroxyl protecting group is selected from the group consisting of silicyl, triphenylmethyl or substituted triphenylmethyl, benzyl or substituted benzyl, alkoxylmethyl or methyl substituted by alkoxyl, acyl, allyl or substituted allyl or alkoxycarbonyl.

26. A process according to claim 21, wherein said hydroxyl protecting group is selected from the group consisting of silicyl, triphenylmethyl or substituted triphenylmethyl, benzyl or substituted benzyl, alkoxylmethyl or methyl substituted by alkoxyl, acyl, allyl or substituted allyl or alkoxycarbonyl.

27. A compound of formula (2) or salts thereof: 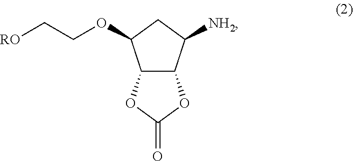
Wherein said R is H or hydroxyl protection group.

28. A compound of formula (2) or salts thereof according to claim 23, wherein said hydroxyl protecting group is selected from the group consisting of silicyl, triphenylmethyl or substituted triphenylmethyl, benzyl or substituted benzyl, alkoxylmethyl or methyl substituted by alkoxyl, acyl, allyl or substituted allyl or alkoxycarbonyl.

29. A compound or salts thereof according to claim 23, wherein said a salt of a compound of formula (2) is a salt of a mineral or organic acid.

30. A process for preparing a compound of formula (2) or salts thereof according to any one of claim 27, comprises:
1) reacting a compound of formula (2-b) with triphosgene to obtain the compound of formula (2-c): 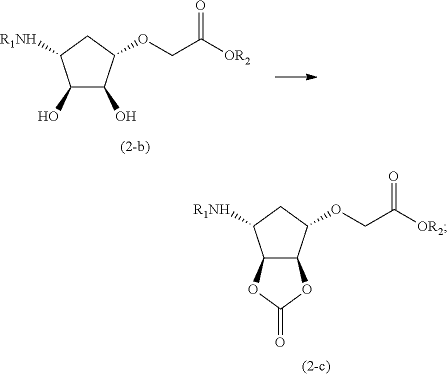
2) reducing the compound of formula (2-c) to obtain the compound of formula (2-d) 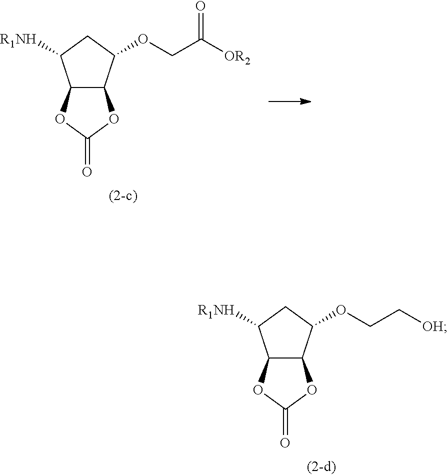
3) deprotecting the compound of formula (2-d); or further to protect the hydroxyl group to obtain the compound of formula (2): 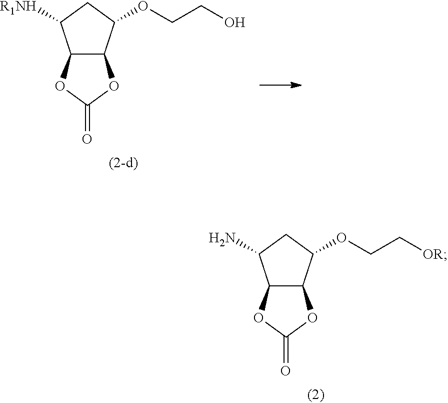
wherein said R₁ is amino protecting group; R₂ is C₁₋₄ alkyl.

31. A process for preparing a compound of formula (2) or salts thereof according to any one of claim 28, comprises:
1) reacting a compound of formula (2-b) with triphosgene to obtain the compound of formula (2-c): 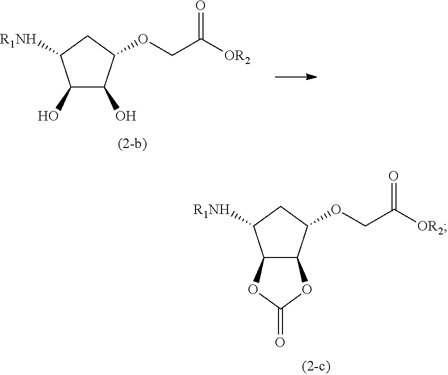
2) reducing the compound of formula (2-c) to obtain the compound of formula (2-d) 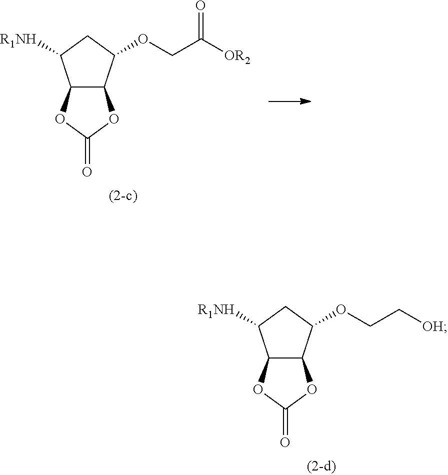
3) deprotecting the compound of formula (2-d); or further to protect the hydroxyl group to obtain the compound of formula (2): 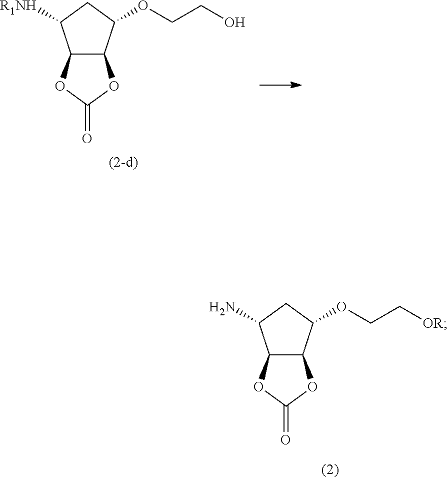
wherein said R₁ is amino protecting group; R₂ is C₁₋₄ alkyl.

32. A process for preparing a compound of formula (2) or salts thereof according to any one of claim 29, comprises:
1) reacting a compound of formula (2-b) with triphosgene to obtain the compound of formula (2-c): 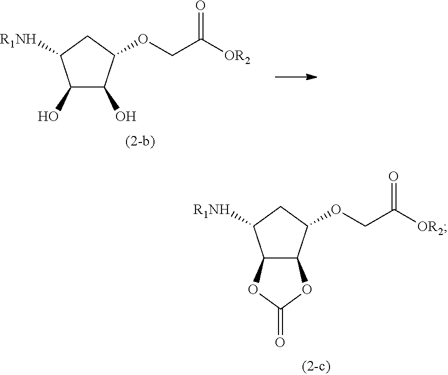
2) reducing the compound of formula (2-c) to obtain the compound of formula (2-d) 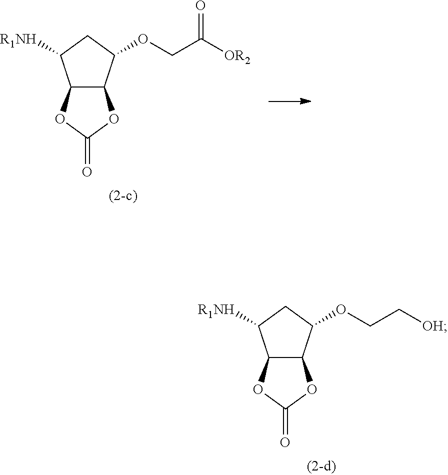
3) deprotecting the compound of formula (2-d); or further to protect the hydroxyl group to obtain the compound of formula (2): 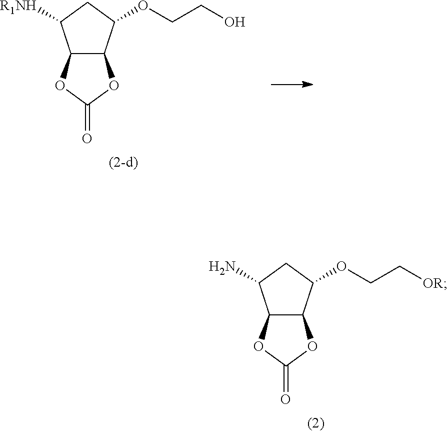
wherein said R₁ is amino protecting group; R₂ is C₁₋₄ alkyl.

33. A process according to claim 30, wherein said R₁ is carboxybenzyl (Cbz), tert-butyloxycarboxyl (BOC), benzyl (Bn), p-methoxyphenyl (PMP) or 9-fluorenylmethyloxycarbonyl (FMOC).

34. A process according to claim 30, wherein said R₂ is methyl or ethyl.

35. A process according to claim 30, wherein said the compound of formula (2-b) is prepared by deprotecting the compound of formula (2-a):

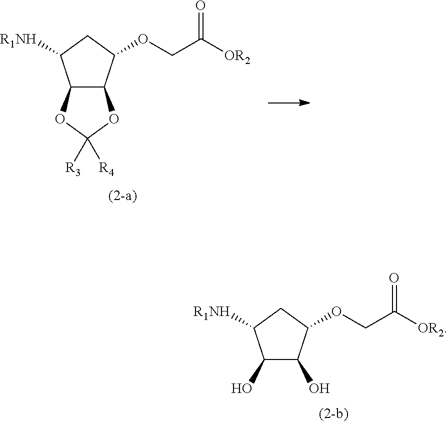

36. A compound of formula (2) or salts thereof for use in the preparation of triazolo pyrimidine compound, 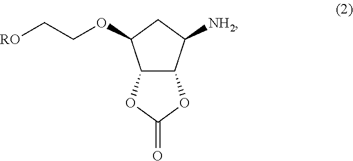
Wherein, said R is H or hydroxyl protecting group.

37. The compound or salts according to claim 36, wherein said triazolo pyrimidine compound is Ticagrelor.

38. An intermediate compound of formula (1-b), (1-c), (1-d) or (1-e): [chemical structure]
